(19) United States
(12) Patent Application Publication
(10) Pub. No.: US 2007/0269437 A1
(43) Pub. Date: Nov. 22, 2007

(54) Pro-Inflammatory and Anti-Inflammatory Antibodies Against the Heparin-Binding Protein (Hbp)

(75) Inventors: Rene Djurup, Gentofte (DK); Hans Jakob Flodgaard, Hellerup (DK); Kjeld Norris, Charlottenlund (DK)

Correspondence Address: BROWDY AND NEIMARK, P.L.L.C.;624 NINTH STREET, NW, SUITE 300, WASHINGTON, DC, 20001-5303, US

(73) Assignee: Leukotech A/S, Copenhagen O (DK)

(21) Appl. No.: 10/572,603

(22) PCT Filed: Sep. 17, 2004
(86) PCT No.: PCT/DK04/00634

§ 371 Date: Mar. 8, 2007

(30) Foreign Application Priority Data
Sep. 19, 2003 (DK) PA 2003 01369

(51) Int. Cl. A61K 39/395; A61P 29/00; C12P 21/08

(52) U.S. Cl. 424/139.1; 435/70.1; 530/387.9; 530/388.1

(57) ABSTRACT

The present invention relates to providing antibodies against human heparin-binding protein (hHBP) or against a homologue of hHBP, such as porcine heparin binding protein (pHBP) or human neutrophil elastase (hNEL), and using said antibodies for the manufacture of a medicament for treatment of Gram positive and/or Gram negative infections, sepsis, disseminated intravascular coagulation, modulation of inflammatory response, and/or prevention of cell apoptosis. Antibodies of the invention are capable of modulating at least one inflammatory response associated with hHBP, in particular, the invention relates to monoclonal antibody F19A5B1, capable of stimulating said at least one inflammatory response, and monoclonal antibody F19A5B4, capable of inhibiting said at least one inflammatory response.

### TECHNICAL FIELD OF THE INVENTION

The present invention relates to providing antibodies against human heparin binding protein (hHBP) and using said antibodies for the manufacture of a medicament for treatment of Gram positive and/or Gram negative infections, sepsis, disseminated intravascular coagulation, modulation of inflammatory response, and/or prevention of cell apoptosis.

### BACKGROUND OF THE INVENTION

A local infection or injury in any tissue rapidly attracts white blood cells into the affected region as part of the inflammatory response, which helps fight the infection or heal the wound. The inflammatory response is complex and is mediated by a variety of signalling molecules produced locally by different types of cells. Some of these molecules act on nearby capillaries, causing the endothelial cells to adhere less tightly to one another but making their surfaces adhesive to passing white blood cells. Other molecules act as chemoattractants for specific types of blood cells, such as monocytes, causing these cells to become polarised and crawl toward the source of the attractant.

White blood cells, specifically polymorphonuclear leukocytes (PMNs), produce a large variety of peptides involved in the inflammatory response. Among these peptides is the heparin-binding protein (HBP), which was first isolated from azurophile granules of human PMNs. A highly homologous peptide was also isolated from PMNs of porcine origin and has been named porcine heparin-binding protein (pHBP) (Flodgaard et al., 1991, Eur. J. Biochem. 197: 535-547; Pohl et al., 1990, FEBS Lett. 272: 200 ff.) HBP has otherwise been termed CAP37 (WO 91/00907, U.S. Pat. Nos. 5,458,874 and 5,484,885) and azurocidin (Wilde et al. 1990, J. Biol. Chem. 265:2038-41).

Sequence analysis of HBP has revealed that the protein bears many similarities to serine proteases, which are important in inflammatory processes, e.g. neutrophil elastase (47% homology) or protease 3 (43% homology), however HBP lacks protease activity due to mutations of two of three amino acids in the highly conserved catalytic triad. The structure of HBP appears from WO 89/08666 and Flodgaard et al., 1991 (Eur. J. Biochem. 197: 535-547).

HBP was originally studied because of its antibiotic and lipopolysaccharide binding properties (Gabay et al., 1989, Proc. Natl. Acad. Sci. U.S.A. 86:5610-5614 and Pereira et al., 1993, Proc. Natl. Acad. Sci. USA 90: 4733-7). However, a number of experimental evidence now supports the concept that HBP is a multifunctional protein, and, in addition to its bactericidal role, is involved during the progression of inflammation due to its effect on the recruitment and activation of monocytes (Pereira et al., 1990, J. Clin. Invest. 85:1468-1476, and Rasmussen et al., 1996, FEBS Lett. 390:109-112), recruitment of T cells (Chertov et al., 1996, J. Biol. Chem. 271:2935-2940), as well as on the induced contraction of endothelial cells and fibroblasts (Ostergaard and Flodgaard, 1992, J. Leuk. Biol. 51:316-323). Ostergaard and Flodgaard (op. cit.) also disclose increased survival of monocytes treated with HBP. Furthermore, in animal models of fecal peritonitis, HBP treatment has been shown to rescue mice from an otherwise lethal injury (Mercer-Jones et al., 1996, In: Surgical Forum, pp. 105-108; Wickel et al., 1997, In: 4th International Congress on the Immune Consequences of Trauma, Chock and Sepsis, Munich, Germany, pp. 413-416).

It would be advantageous to develop new medicaments aimed to modulate the biological responses mediated by the HBP, in particular inflammatory response, using new agoinists or antagonists of the protein.

### SUMMARY OF THE INVENTION

It was surprisingly found that monoclonal antibodies against human HBP (hHBP) are capable of modulating inflammatory response. Even more surprisingly, it was found that the antibodies produced by one clone are potent stimulators of the HBP-mediated inflammatory response, while the antibody produced by another clone do indeed reduce or even inhibit said response.

Accordingly, the present invention relates in one aspect to a pharmaceutical composition for the stimulating of at least one inflammatory response associated with hHBP or a homologue thereof, comprising an antibody against hHBP (SEQ ID NO: 1), said antibody capable of binding to an epitope within the sequence comprising amino acid residues 1 to 19 or 45 to 226 according to SEQ ID NO: 1 and thereby stimulating at least one inflammatory response mediated by hHBP.

Another aspect of the invention concerns a pharmaceutical composition for the inhibiting of at least one inflammatory response associated with human heparin binding protein (hHBP), or a homologue thereof, comprising an antibody against hHBP (SEQ ID NO: 1), said antibody capable of binding to an epitope within the sequence comprising amino acid residues 20 to 44 according to SEQ ID NO: 1 and thereby inhibiting at least one inflammatory response mediated by hHBP.

The invention discloses pro-inflammatory anti-hHBP monoclonal antibody produced by clone F19A5B1 (ECACC Ass. No.: 03090301) and anti-inflammatory anti-hHBP monoclonal antibody produced by clone F19A5B4 (ECACC Ass. No.: 03090302). The invention also relates to a method for producing the above antibodies and cells producing thereof.

Other aspects of the invention concern uses of the pharmaceutical compositions comprising anti-hHBP antibodies for the stimulation or inhibition of the inflammatory response. The invention also relates to uses antibodies F19A5B1 and F19A5B4 for (1) stimulating or inhibiting inflammatory response and/or (2) manufacture of a medicament for the treatment individuals having suppressed immune system, cancer, autoimmune diseases and/or trauma or individuals having a sustained inflammatory response.

The invention also relates to recombinant antibody molecules, antibody fragments, and other compounds, said molecules, fragments and compounds being capable of recognising the epitopes of the invention.

### DETAILED DESCRIPTION OF THE INVENTION

1. Inflammation

The present invention relates to providing antibodies and using said antibodies for the manufacture of a medicament for modulation of the inflammatory response.

Inflammation is a defense reaction caused by tissue damage due to a mechanical injury or bacterial, virus or other organism infection. The inflammatory response involves three major stages: first, dilation of capillaries to increase blood flow; second, microvascular structural changes and escape of plasma proteins from the bloodstream; and third, leukocyte transmigration through endothelium and accumulation at the site of injury and infection. The inflammatory response begins with a release of inflammatory mediators. Inflammatory mediators are soluble, diffusible molecules that act locally at the site of tissue damage and infection, and at more distant sites, influencing consequent events of the inflammatory response. Inflammatory mediators can be exogenous, e.g. bacterial products or toxins, or endogenous, which are produced within the immune system itself, as well as injured tissue cells, lymphocytes, mast cells and blood proteins.

In one aspect the present invention relates to an inflammatory response in connection with bacterial infection.

By “bacterial infection” in the present context is meant the invasion of normally sterile host tissue by bacteria. Bacterial infection of the invention may be due to invasion of either Gram negative or Gram positive bacteria, or a combination thereof or other infectious agents including fungi and virus. In one embodiment the present invention relates to the inflammatory response due invasion of Gram negative bacteria selected from the group comprising Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, Chromobacterium

In another embodiment the invention relates to an inflammatory response due to invasion by Gram positive bacteria selected from the group comprising Bacillaceae, Micrococcaceae (for example Staphylococcus aureus), Mycobacteriaceae (for example Staphylococcus pneumoniae), Peptococcaceae.

In an additional another embodiment the invention relates to an inflammatory response associated with sepsis, severe sepsis and/or septic shock.

By “sepsis” in the present context is meant the systematic inflammatory response to bacterial infection, characterised by one or more of the following conditions as a result of infection: temperature>38° C. or <36° C., heart rate>90 beats/min, respiratory rate>20 breaths/min or PaCO₂<32 torr (<4.3 kPa), and WBC>12 000 cells/mm³ or <4000 cells/mm³ or 10% immature (band) forms.

By “severe sepsis” in the present context is meant sepsis associated with organ dysfunction, hypoperfusion, or hypotension, hypoperfusion and hypotension abnormalities may include, but are not limited to, lactic acidosis (acidic condition in blood), oliguria (meaning reduction in urine production), or acute alteration in mental status.

By “septic shock” in the present context is meant sepsis with hypotension despite adequate fluid resuscitation, along with the presence of perfusion abnormalities that may include, but are not limited to, lactic acidosis, oliguria, or acute alteration in mental status.

In yet another embodiment the invention relates to an inflammatory response associated with disseminated intravascular coagulation (DIC).

By “DIC” in the present context is meant a pathophysiologic condition involving a continuum of events that occur in the coagulation pathway in association with a variety of well-defined clinical situations, including sepsis, major trauma, and abruptio placenta, and with laboratory evidence of the following: procoagulant activation, fibrinolytic activation, inhibitor consumption and biochemical evidence of end-organ damage or failure.

The present invention features pharmaceutical compositions comprising compounds capable of modulating at least one inflammatory response associated with the above pathological conditions.

In the present context by the term “inflammatory response” is meant a biological process or a series of biological reactions specifically associated with inflammation. The invention concerns the inflammatory response due to biological activity of hHBP and selected from the group comprising
i) up-regulation of the gene expression in the immune cells, preferably monocytes/macrophages, leading to secretion of endogenous inflammatory mediators including receptors for inflammatory mediators and transcription factors involved in the signal tranduction of the inflammatory mediators, said mediators being preferably selected from the group comprising cytokines, selected from the group TNFalpha IL-1, IL-6, G-CSF, GM-CSF, M-CSF. Chemokines selected from the group comprising IL-8, MCP-1, receptors selected from the group Tissue factor and IL-2Ralpha,
ii) activation of the production of bradykinin by the phase contact system,
iii) increasing the blood concentration of monocytes and/or local accumulation thereof at the sites of inflammation,
iv) increasing the life-time of monocytes, neutrophils and other immune cells due to inhibition of apoptosis,
v) activation of expression of adhesion molecules by the vascular endothelial cells, said adhesion molecules being preferably selected from the group comprising PECAM, ICAM-1, E-selectins, VCAM-1,
vi) activation of the contact phase system producing bradykinin leading to an increased vascular permeability,
vii) increasing the phagocytic potential of monocytes/macrophages,
viii) up-regulation of class-II MHC.

By the term “modulating” of an inflammatory response is meant a capability of a compound to increase or decrease the threshold of the response. It is understood that the compound is capable of stimulating of inflammatory response when the threshold/upper limit of the response is increased in connection with using of the compound, and the compound is capable of inhibiting of inflammatory response when the threshold of the response is decreased.

In one aspect a pharmaceutical composition of the invention comprises a compound capable of stimulating at least one inflammatory response, a pro-inflammatory compound.

In another aspect the present invention relates to a pharmaceutical composition comprising a compound capable of inhibiting at least one inflammatory response, an anti-inflammatory compound.

The present invention in a preferred embodiment concerns at least one of the above inflammatory responses mediated by hHBP.

Pro-Inflammatory Compound

In one aspect the invention relates to a pro-inflammatory compound being an antibody against hHBP.

The pro-inflammatory antibody of the invention is an antibody, which is
i) capable to recognise an epitope located within a sequence comprising amino acid residues 1 to 19 or 45 to 225 according to the sequence identified in SEQ ID NO:1;
ii) capable of stimulating the inflammatory response associated with one or more biological activities of hHBP or a hHBP fragment comprising amino acids 20-44 according to the sequence identified as SEQ ID NO: 1,
i) capable of stimulating at least one of the above described inflammatory responses, wherein the antibody are often capable of stimulating any two or more of the above responses.

A pro-inflammatory antibody of the invention may in one embodiment be capable of stimulating at least one of the above described inflammatory responses in synergistic action with bacterial products including, but not limited to LPS (Lipopolysaccharide), PGN (peptidoglycan), LTA (Lipotechoic acid), MDP (muramyldipeptide) and PCW (purified cell wall from bacteria) present in the blood or in a biological sample collected from an individual. In another embodiment, a pro-inflammatory antibody of the invention is capable of stimulating at least one of the above described inflammatory responses in the absence of the above mentioned bacterial products.

The pro-inflammatory antibody may be either polyclonal or monoclonal. In a preferred embodiment the pro-inflammatory antibody is a monoclonal antibody against hHBP. A preferred pro-inflammatory antibody of the invention is the antibody produced by a cell of clone F17A5B1 (ECACC Ass. No.: 03090301)

Another preferred pro-inflammatory antibody of the invention is the antibody that is capable of stimulating the production of cytokine IL-6.

In another aspect the invention relate to a fragment of the above antibody, said fragment being capable of the features of a pro-inflammatory antibody of above, such as a capability of binding to an epitope of hHBP or a hHBP homologue, said epitope being specifically recognisable by said pro-inflammatory antibody, and stimulating at least one of the inflammatory responses describes above.

Still, in another aspect the invention concerns a recombinant protein, said recombinant protein comprising a fragment of the above pro-inflammatory antibody, or a full-length antibody molecule of above, and which is capable of activity of said fragment or said antibody molecule.

An antibody, which is capable of binding to the epitope of hHBP specifically recognisable by the above pro-inflammatory antibody, by a fragment thereof or a recombinant protein thereof, is also in the scope of the invention as a pro-inflammatory compound capable of stimulating at least one of the above described inflammatory responses. It is understood that the latter antibody is an antibody against another antigen then hHBP, or hHBP homologue (e.g. pHBP or hNEL), said antibody, however, are capable of binding to an epitope of the invention and stimulating inflammatory response.

Anti-inflammatory Compound

It is another important objective of the invention to provide a new anti-inflammatory compound, which is capable of serving as an inhibitor of the sustained inflammatory response.

The continuous presence of inflammatory mediators, such as for example TNF alpha in the body in response to sustained presence of bacterial products or even live bacteria locally during days or weeks following trauma and/or infection promotes the reactions to inflammation, such as, for example, heat, swelling, and pain. The sustained inflammatory response has been proven to be very harmful to the body. If the bacterial products or live bacteria become spread universally in the body from their local focus the inflammatory reaction becomes overwhelming and out of control and leads to sepsis which eventually progress further to severe sepsis and septic shock. Anti-inflammatory peptides may be used to block or suppress the overwhelming sustained inflammatory response represented by a massive and harmful cytokine cascade in the blood and vital organs such as lung, liver intestine, brain and kidneys.

In the present context by the term “anti-inflammatory compound” is meant a compound which is capable of
i) decreasing or inhibiting the gene expression in the immune cells, preferably monocytes/macrophages in response to bacterial products, live bacteria or trauma to produce endogenous inflammatory mediators including receptors for inflammatory mediators and transcription factors involved in the signal transduction of the inflammatory mediators, said mediators being preferably selected from the group comprising cytokines, selected from the group TNFalpha IL-1, IL-6, G-CSF, GM-CSF, M-CSF, chemokines selected from the group comprising IL-8, MCP-1, or receptors selected from the group Tissue factor and IL-2Ralpha. and/or
ii) decreasing or inhibiting the production bradykinin by the phase contact system, and/or;
iii) decreasing or inhibiting the chemoattraction of monocytes to the sites of inflammation, and/or
iv) decreasing or inhibiting the expression of the adhesion molecules by vascular endothelial cells, said adhesion molecules being preferably selected from the group comprising PECAM, ICAM-1, E-selectins, VCAM-1 and/or
v) decreasing or inhibiting the activation of the contact phase system to produce bradykinin leading to increased vascular permeability, and/or
vi) stimulating the synthesis of an anti-inflammatory mediator selected from the group of IL-10 and IL-12, and/or
vii) decreasing the concentration of endotoxin in body fluids of septic patients, and/or

The present invention relates in one aspect to the anti-inflammatory compound being an antibody against hHBP, or a hHBP homologue, such as for example porcine HBP (pHBP) or human neutrophil elastase (hNEL),
i) capable of recognising an epitope located within a sequence comprising amino acid residues 20 to 44 according to the sequence identified in SEQ ID NO:1;
ii) capable of inhibiting the inflammatory response associated with one or more biological activities of hHBP or a hHBP fragment comprising amino adds 20-44 according to the sequence identified as SEQ ID NO: 1,
iii) capable of at least one of the above activities of an anti-inflammatory compound, more preferable at least two of the above activities, even more preferable at least tree of the above activities, even more preferable at least four of the above activities, even more preferable at least five of the above activities, even more preferable at least six of the above activities, even more preferable at least seven of the below activities, and most preferably eight of the above activities.

In a preferred embodiment the invention relates to an anti-inflammatory antibody capable of inhibiting the secretion of cytokine IL-6 from monocytes in response to bacterial products including, but not limited to, LPS (Lipopolysaccharide), PGN (peptidoglycan), LTA (Lipotechoic acid), MDP (muramyldipeptide) and PCW (purified cell wall from bacteria). In another preferred embodiment the invention relates to an anti-inflammatory antibody capable of decreasing or inhibiting the secretion of cytokine IL-6 from monocytes activated by HBP, a HBP fragment comprising amino acid residues 20-44 in the absence of the mentioned bacterial products, and/or in the absence of other monocyte activating factors and/or conditions such as for example conditions of trauma, auto-immune disease, cancer, sepsis, severe sepsis, septic shock and/or disseminated intravascular coagulation.

The anti-inflammatory antibody may be either a polyclonal or monoclonal antibody. In a preferred embodiment the anti-inflammatory antibody of the invention is a monoclonal antibody against hHBP. A preferred monoclonal antibody of the invention is the antibody produced by a cell of clone F17A5B4 (ECACC Ass. No.: 03090302)

In another aspect the invention relates to a fragment of the above anti-inflammatory antibody, said fragment being capable of the features of the anti-inflammatory anti-bodies of above, such as a capability of binding to an epitope of hHBP or a hHBP homologue, and thereby inhibit at least one of the inflammatory responses describes above, said epitope being specifically recognisable by the anti-inflammatory anti-body of above.

Still in another aspect the invention concerns a recombinant protein, which comprises a fragment of the above anti-inflammatory antibody, or the full-length anti-inflammatory antibody molecule of above, and which is capable of activity of said fragment or antibody molecule.

Antibody molecules specific for other antigens then hHBP, or the hHBP homologues of the invention, which, however, are capable of binding to the epitope of hHBP, which specifically recognised by the above anti-inflammatory antibody, fragment thereof or recombinant protein comprising thereof, are also in the scope of the invention as anti-inflammatory compounds capable of inhibiting at least one of the inflammatory responses mediated by hHBP.

2. Antibody

It is an objective of the present invention to provide an antibody capable of selectively binding to hHBP (SEQ ID NO: 1), a fragment thereof, recombinant protein thereof or homologue thereof, such as for example pHBP (SEQ ID NO: 588) or hNEL (SEQ ID NO: 589).

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immuno-specific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from anti-body fragments. As used herein, “functional fragment” with respect to antibodies, refers to Fv, F(ab) and F(ab′)₂ fragments.

Antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it is has similar or immunological properties relative to antibody that binds with specificity to an epitope located within a sequence selected from the group consisting of SEQ ID NO: 1, 588 and 589, such as an epitope comprising at least one of the sequences identified in SEQ ID NO: 2-587, for example such as an epitope located within a sequence comprising amino acid residues 1 to 19 or 45 to 225 according to the sequence identified in SEQ ID NO:1.

Antibody fragments retain some ability to selectively bind with its antigen or receptor. Some types of antibody fragments are defined as follows:
(1) Fab is the fragment that contains a monovalent antigen-binding fragment of an antibody molecule. A Fab fragment can be produced by digestion of whole antibody with the enzyme papain to yield an intact light chain and a portion of one heavy chain.
(2) Fab′ is the fragment of an antibody molecule can be obtained by treating whole antibody with pepsin, followed by reduction, to yield an intact light chain and a portion of the heavy chain. Two Fab′ fragments are obtained per anti-body molecule.

Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction.
(4) F(ab′)₂ is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.
(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies 113: 269-315 Rosenburg and Moore eds. Springer-Verlag, NY, 1994.

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

The preparation of polyclonal antibodies is well-known to those skilled in the art. See, for example, Green et al. 1992. Production of Polyclonal Antisera, in: Immuno-chemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1, which are hereby incorporated by reference.

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal anti-bodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG). In: Methods in Molecular Biology, 1992, 10:79-104, Humana Press, NY. ( ).

Methods of in vitro and in vivo manipulation of monoclonal antibodies are well known to those skilled in the art. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage anti-body libraries using the techniques described in Clackson et al., 1991, Nature 352: 624-628, as well as in Marks et al., 1991, J Mol Biol 222: 581-597. Another method involves humanizing a monoclonal antibody by recombinant means to generate antibodies containing human specific and recognizable sequences. See, for review, Holmes, et al., 1997, J Immunol 158:2192-2201 and Vaswani, et al., 1998, Annals Allergy, Asthma & Immunol 81:105-115.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in anti-bodies derived from another species or belonging to another antibody class or subclass, as well as fragments of such antibodies, so long as they exhibit the desired biological activity (U.S. Pat. No. 4,816,567); Morrison et al., 1984, Proc Natl Acad Sci 81, 6851-6855.

Methods of making antibody fragments are also known in the art (see for example, Harlow and Lane, Antibodies: A Laboratory Manual, Cold Spring Harbor Laboratory, NY, 1988, incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., 1991, In: Methods: A Companion to Methods in Enzymology, 2:97; Bird et al., 1988, Science 242:423-426; U.S. Pat. No. 4,946,778; and Pack, et al., 1993, BioTechnology 11:1271-77. Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., 1986, Nature 321, 522-525; Reichmann et al., 1988, Nature 332, 323-329; Presta, 1992, Curr Op Struct Biol 2:593-596; Holmes et al., 1997, J Immunol 158:2192-2201 and Vaswani, et al., 1998, Annals Allergy, Asthma & Immunol 81:105-115.

The invention provides both polyclonal and monoclonal antibodies. The generation of antibodies may be achieved by standard methods of the art for producing polyclonal and monoclonal antibodies using a natural or recombinant HBP polypeptide or fragment thereof as an antigen. Such antibodies would be in a preferred embodiment generated using a naturally occurring or recombinantly produced hHBP, or hHBP-related polypeptides, namely pHBP and hNEL, having amino acid sequences set forth in SEQ ID NO: 1, 588 or 589 correspondingly, or variants or fragments thereof, or, in a more preferred embodiment, using fragments of said polypeptides, wherein said fragments would meet at least two of the following criteria:
(i) being a natural or synthetic contiguous amino acid sequence of at least 8 amino acids derived from any of the sequences identified as SEQ ID NOS: 1, 588 or 589;
(ii) being a natural or synthetic contiguous amino acid sequence of at least 8 amino acids comprising at least one amino acid sequence selected from any of the sequences identified as SEQ ID NOS: 2-587.

The antibodies may also be produced in vivo by the individual to be treated, for example, by administering an immunogenic fragment according to the invention to said individual. Accordingly, the present invention further relates to a vaccine comprising an immunogenic fragment described above.

In one preferred embodiment the invention relates to an antibody, or antibody fragment, capable of binding to an epitope comprising a fragment of the amino acid sequence set forth SEQ ID NO: 1, said fragment consisting of the amino acid residues 1 to 19 or 45 to 225 according to SEQ ID NO: 1. Preferably that the epitope is located within the sequence hHBP (SEQ ID NO: 1).

In another preferred embodiment the invention relates to an antibody, capable of binding to an epitope comprising a fragment of the amino acid sequence set forth SEQ ID NO: 1, said fragment consisting of the amino acid residues 20 to 44 according to SEQ ID NO: 1. Preferably that the epitope is located within the sequence of hHBP (SEQ ID NO: 1).

It is understood that the latter antibodies are raised against hHBP, a fragment of hHBP or hHBP homologue. In particular, the invention concerns a monoclonal anti-body against hHBP. In one preferred embodiment the monoclonal antibody is anti-body F19A5B1. According to the invention antibody F19A5B1 is capable of binding to an epitope comprising one or more amino acid residues of the sequence consisting of amino acid residues 1 to 19 or 45 to 225 of the sequence set forth in SEQ ID NO: 1, said antibody is capable of stimulating at least one inflammatory response associated with one or more biological activities of hHBP or a hHBP fragment comprising amino acids 20-44 according to the sequence identified as SEQ ID NO: 1. In another preferred embodiment the monoclonal antibodies is antibody F19A5B4. According to the invention antibody F19A5B4 is capable of binding to an epitope comprising one or more amino acid residues of the sequence consisting of amino acid residues 20 to 44 of the sequence set forth in SEQ ID NO: 1, said antibody is capable of inhibiting at least one inflammatory response associated with one or more biological activities of hHBP or a hHBP fragment comprising amino acids 20-44 according to the sequence identified as SEQ ID NO: 1.

In still another embodiment, the invention relates to an antibody capable of recognising an epitope of the above embodiments, wherein said antibody is not anti-hHBP antibody. An example of such antibody may be an antibody raised against recombinant or natural protein or peptide, said protein or peptide comprising an amino acid sequence comprising the epitope(s) described above.

In another aspect, the application relates to a method for producing an antibody of the invention, said method comprising a step of providing hHBP or an immunogenic fragment of hHBP, or related immunogenic peptides/polypeptides described above, and using the provided immunogens for the production of an antibody capable of modulating of inflammatory response by any of the methods described above.

Production of Immunogenic Peptide Sequences and/or Antibody Fragments

The immunogenic molecules comprising hHBP, pHBP, hNLA and fragments thereof, of the present invention, or antibody and fragments thereof may be prepared by conventional synthetic methods, recombinant DNA technologies, enzymatic cleavage of full-length proteins which the peptide sequences are derived from, or a combination of said methods.

1. Synthetic Preparation

The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

Peptides may for example be synthesised by using Fmoc chemistry and with Acm-protected cysteines. After purification by reversed phase HPLC, peptides may be further processed to obtain for example cyclic or C- or N-terminal modified isoforms. The methods for cyclization and terminal modification are well-known in the art and described in detail in the above-cited manuals.

In a preferred embodiment, immunogenic peptide sequences of the invention are produced synthetically, in particular, by the Sequence Assisted Peptide Synthesis (SAPS) method.

2. Recombinant Production

The DNA sequence encoding a immunogenic peptide or full-length protein of the invention, or an antibody molecule of the invention may be prepared synthetically by established standard methods, e.g. the phosphoamidine method described by Beaucage and Caruthers, 1981, Tetrahedron Lett. 22:1859-1869, or the method described by Matthes et al., 1984, EMBO J. 3:801-805. According to the phosphoamidine method, oligonucleotides are synthesized, e.g. in an automatic DNA synthesizer, purified, annealed, ligated and cloned in suitable vectors.

The DNA sequence encoding a peptide may also be prepared by fragmentation of the DNA sequences encoding the corresponding full-length protein of peptide origin, using DNAase I according to a standard protocol (Sambrook et al., Molecular cloning: A Laboratory manual. 2 rd ed., CSHL Press, Cold Spring Harbor, N.Y., 1989). The present invention relates to full-length proteins selected from the groups of proteins identified above. The DNA encoding the full-length proteins of the invention may alliteratively be fragmented using specific restriction endonucleases. The fragments of DNA are further purified using standard procedures described in Sambrook et al., Molecular cloning: A Laboratory manual. 2 rd ed., CSHL Press, Cold Spring Harbor, N.Y., 1989.

The DNA sequence encoding a full-length protein may also be of genomic or cDNA origin, for instance obtained by preparing a genomic or cDNA library and screening for DNA sequences coding for all or part of the full-length protein by hybridisation using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primer, for instance as described in U.S. Pat. No. 4,683,202 or Saiki et al., 1988, Science 239:487-491.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding a peptide or a full-length protein should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the SV 40 promoter (Subramani et al., 1981, Mol. Cell Biol. 1:854-864), the MT-1 (metallothionein gene) promoter (Palmiter et al., 1983, Science 222: 809-814) or the adenovirus 2 major late promoter. A suitable promoter for use in insect cells is the polyhedrin promoter (Vasuvedan et al., 1992, FEBS Lett. 311:7-11). Suitable promoters for use in yeast host cells include promoters from yeast glycolytic genes (Hitzeman et al., 1980, J. Biol. Chem. 255:12073-12080; Alber and Kawasaki, 1982, J. Mol. Appl. Gen. 1: 419-434) or alcohol dehydrogenase genes (Young et al., 1982, in Genetic Engineering of Microorganisms for Chemicals, Hollaender et al, eds., Plenum Press, New York), or the TPI1 (U.S. Pat. No. 4,599,311) or ADH2-4c (Russell et al., 1983, Nature 304:652-654) promoters. Suitable promoters for use in filamentous fungus host cells are, for instance, the ADH3 promoter (McKnight et al., 1985, EMBO J. 4:2093-2099) or the tpiA promoter.

The coding DNA sequence may also be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) promoters. The vector may further comprise elements such as polyadenylation signals (e.g. from SV 40 or title adenovirus 5 Elb region), transcriptional enhancer sequences (e.g. the SV 40 enhancer) and translational enhancer sequences (e.g. the ones encoding adenovirus VA RNAs).

The recombinant expression vector may further comprise a DNA sequence enabling the vector to replicate in the host cell in question. An example of such a sequence (when the host cell is a mammalian cell) is the SV 40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hydromycin or methotrexate.

The procedures used to ligate the DNA sequences coding the peptides or full-length proteins, the promoter and the terminator, respectively, and to insert them into suitable vectors containing the information necessary for replication, are well known to persons skilled in the art (cf., for instance, Sambrook et al., op.cit.).

To obtain recombinant peptides of the invention the coding DNA sequences may be usefully fused with a second peptide coding sequence and a protease cleavage site coding sequence, giving a DNA construct encoding the fusion protein, wherein the protease cleavage site coding sequence positioned between the HBP fragment and second peptide coding DNA, inserted into a recombinant expression vector, and expressed in recombinant host cells. In one embodiment, said second peptide selected from, but not limited by the group comprising glutathion-S-reductase, calf thymosin, bacterial thioredoxin or human ubiquitin natural or synthetic variants, or peptides thereof. In another embodiment, a peptide sequence comprising a protease cleavage site may be the Factor Xa, with the amino acid sequence IEGR, enterokinase, with the amino acid sequence DDDDK, thrombin, with the amino acid sequence LVPR/GS, or Acharombacter lyticus, with the amino acid sequence XKX, cleavage site.

The host cell into which the expression vector is introduced may be any cell which is capable of expression of the peptides or full-length proteins, and is preferably a eukaryotic cell, such as invertebrate (insect) cells or vertebrate cells, e.g. Xenopus laevis oocytes or mammalian cells, in particular insect and mammalian cells. Examples of suitable mammalian cell lines are the HEk293 (ATCC CRL-1573), COS (ATCC CRL-1650), BHK (ATCC CRL-1632, ATCC CCL-10) or CHO (ATCC CCL-61) cell lines. Methods of transfecting mammalian cells and expressing DNA sequences introduced in the cells are described in e.g. Kaufman and Sharp, J. Mol. Biol. 159, 1982, pp. 601-621; Southern and Berg, 1982, J. Mol. Appl. Genet. 1:327-341; Loyter et al., 1982, Proc. Natl. Acad. Sci. USA 79: 422-426; Wigler et al., 1978, Cell 14:725; Corsaro and Pearson, 1981, in Somatic Cell Genetics 7, p. 603; Graham and van der Eb, 1973, Virol. 52:456; and Neumann et al., 1982, EMBO J. 1:841-845.

Alternatively, fungal cells (including yeast cells) may be used as host cells. Examples of suitable yeast cells include cells of Saccharomyces spp. or Schizosaccharomyces spp., in particular strains of Saccharomyces cerevisiae. Examples of other fungal cells are cells of filamentous fungi, e.g. Aspergillus spp. or Neurospora spp., in particular strains of Aspergillus oryzae or Aspergillus niger. The use of Aspergillus spp. for the expression of proteins is described in, e.g., EP 238 023.

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be prepared according to published recipes (e.g. in catalogues of the American Type Culture Collection).

The peptides or full-length proteins recombinantly produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. HPLC, ion exchange chromatography, affinity chromatography, or the like.

3. Screening Assays

Antibodies provided by any of the above described methods are preferably aimed for the manufacture of a medicament for the treatment of a pathological condition, wherein inhibiting or stimulating of inflammatory response is required. Accordingly, produced antibodies are further screened for their biological activity to be selected as useful for the purpose of the invention.

In the present content by “biological activity” is meant that a capability to demonstrate at least one of the following biological activities: (1) stimulating/inhibiting the production of IL-1, IL-6, IL-8, GCSF, GM-CSF, M-CSF, TNF-α, MCP-1, group Tissue factor, IL-2R-α; (2) enhancing/decreasing the bactericidal, chemotactic and/or anti-apoptotic activity of hHBP, (3) stimulating inhibiting the vascular permeability, expression of adhesion molecules PECAM or ICAM1 by endothelial cells, and/or production of bradykinin dependent on hHBP; (4) stimulating/inhibiting the phagocytic potential of the cells, and/or (5) up/down-regulating class-II MHC.

In one preferred embodiment an antibody of the invention is able to demonstrate at least one of the above activities. In another embodiment, an antibody is capable of two or more activities of above.

Methods for evaluating of the above listed biological activities of the antibodies according to the invention are well known in art, and a number of assays for evaluating the biological activity of the antibody are available at the present.

One of such assays useful for the evaluation of chemotactic activity may for example be a method according to Cates et al. (in Leukocyte chemotaxis, p 67. Gallin and Quie eds, Raven Press, NY, 1978), or Keire et al. (J. Biol. Chem. 2001, 276: 48847-53).

To evaluate the bactericidal activity of a medicament comprising the antibody the assay described by Shafer et al. (Infect. Immun. 1986, 53:651-55) may be used.

The measuring of cell apoptosis in the presence or in the absence of the present antibodies may for example be done according to Linde et al. (Anal. Biochem. 2000, 280:186-8).

The vascular permeability may be determined by using the assay as described by Gautam et al. in 1998 (Br J Pharmacol 1998 November; 125(5):1109-14)

The changes in expression of different polypeptides, such as for example IL-1, IL-6, IL-8, TNF-α, thrombospondin, PECAM or ICAM in the presence of the antibodies according to the invention may, for example, be evaluated either by reverse phase transcriptase, immunoassay, immunoblotting, or immunostaining of the treated cells grown in culture. Secretion of the mediators of inflammation, such as for example IL-1, IL-6, IL-8, TNF-α, may be evaluated in Whole blood screening assay (WB) described below.

Human whole blood (WB) samples contain besides red cells, platelets and plasma the white blood cells including the neutrophils and monocytes. Neutrophils and monocytes have receptors for bacterial products such as LPS, PGN and LTA. The bacterial products react directly or via specific binding proteins to receptors on the monocytes thereby stimulating them to secrete and release inflammatory cytokines such as IL-1, L-6, and TNF-α. HBP and HBP fragments, specifically the fragments comprising 20-44 aa according to SEQ ID NO: 1 or 2 do not influence the production of cytokines when they present in the blood, however they can significantly amplify cytokine synthesis and secretion induced by bacterial products. In the assay the amplification of 160 μM HBP per ml WB typically leads to at least three-fold amplification of the cytokine secretion.

In the screening assay LPS from the E. coli may be used to stimulate the monocytes in WB (anti-coagulated by use of citrate) to secrete IL-6 and activity of the antibodies may be evaluated in relation to the amount of IL-6 in plasma subsequently separated from WB. The activity may be related to the capability to:
1. increase IL-6 secretion in absence of bacterial products
2. increase IL-6 secretion in presence of bacterial products
3. decrease IL-6 secretion in presence of bacterial products
4. inhibit amplification of IL-6 secretion induced by intact HBP

All operations must be carried out in LAF cabinet by observance of stringent aseptic techniques. All test tubes, pipette tips etc. must be pyrogen-free. Buffers must be prepared by use of sterile, pyrogen water, preferably water for injection. Usage of 0.1% pyrogen-free BSA/PBS for all dilutions is recommended.

The assay may be performed as the following:
Add 20 μl of the antibody (in concentrations from 25 to 2500 μg/ml) to 100 μl freshly drawn (less than 4 hours old) citrate whole blood from a healthy human volunteer.
Add 20 μl bacterial component (LPS, LTA or PGN) in concentrations from 5 to 5000 ng/ml, preferably 50 to 500 ng/ml.
Mix well and incubate for 16-18 hours in an atmosphere of 5% carbon dioxide and at least 95% relative humidity.
At the end of the incubation add at least 5 volumes (700 μl) 0.1% BSA/PBS and mix well.
Centrifuge 10 min. at 10.000 g.
Aspirate 500 μl supernatant.
Determine the level of IL-6 by a specific human immune assay for human IL-6 with sensitivity of at least 3 pg/ml, e.g. Human IL-6 kit from RnD Systems (cat. no. D 6050).

Negative controls may be 100 μl WB plus 40 μl 0.1% BSA/PBS.

Positive control may be 100 μl WB plus 20 μl LPS (same concentration as used for testing the peptide) and 20 μl 0.1% BSA/PBS.

5. Functional Antibody

A number of experimental evidence now supports the concept that HBP is a multifunctional protein, and, in addition to its bactericidal role, is involved during the progression of inflammation due to its effect on the recruitment and activation of monocytes, recruitment of T cells, as well as on the induced contraction of endothelial cells and fibroblasts.

There has been no identification of a HBP receptor, but receptor-like structures or binding sites of HBP have been identified. HBP is a dipole separated by a hydrophobic cleft and it is therefore capable to interact with both positively and negatively charge surfaces and molecules and with hydrophobic molecules and epitomes. The charged surface areas (the epitomes) of HBP are important for several of its functions. Without being bound by theory some of such functions are described below:

It has been demonstrated that HBP's positively charged epitomes bind to negatively charged macromolecules such as the heparan sulphate and chondroitin sulphate side chains of the proteoglycans, which are present at the surface of nearly every adherent mammalian cells. Proteoglycans are proteins with long carbohydrate chains of the glucosaminoglycans (GAG) type attached. They have recently been recognized as an important part of the signaling mechanism between cells. The proteoglycans are today recognized as co-receptors that can influence how e.g. the growth factor interacts with its receptor. Co-receptors affect which signal molecules bind to the receptor, how strong the interaction is or how far the signal spreads. Coreceptors regulate such decisions as when the cell divides, what type of proteins it manufactures and even if it should die. HBP has been shown to bind to the carbohydrate part (e.g. heparan sulphate) of the syndecan family of proteoglycans, which play an important role in internalization of proteins. The binding of HBP to such proteoglycans lead to uptake of HBP into endothelial cells and probably other cell types as well. Heparan sulphate and similar highly charged negative molecules of the glucosaminoglycan type may therefore serve as binding sites for HBP, mediating many of its diverse regulatory functions. In this context it is should be noted that heparan sulphate and similar glucosaminoglycans are not just simple negatively charged molecules mediating a non-specific ionic interaction. In contrast e.g. the heparan sulphate are synthesized such that very diverse and subtle variations in the structure are achieved. Accordingly, the synthesized heparan sulphate molecules may fit only very specific positively charged epitopes, such as the ones found on the surface of HBP. The heparan sulphates and similar proteoglycans with GAG side chains may therefore be seen as a proper receptor or co-receptor for HBP.

HBP may also exploit its dipolar nature by activating the contact phase system. The contact phase system consists of HMWK and three other proteins which are closely bound together on the cell surface. HMWK is a large protein consisting of 6 domains, of which one (domain 4) contains the Bradykinin sequence. An electrostatic binding from a positively charged histidin-rich area in domain 5 of HMWK to negatively charged heparan sulphate and chondroitin sulphate proteoglycans contribute significantly to the binding of HMWK to cell surfaces. The activation of the contact phase system requires that the individual components (HMWK, fXII and pre-kallikrein) are brought in close contact to each other and probably also that certain conformational changes are induced. Heparin-binding protein (HBP) has been shown to play a pivotal role in activating the contact phase system, and to be capable of highly effectively displacing HMWK from GAG in an in vitro model (Renne, T. 1999. This occurs most likely by formation of two electrostatic bindings, one between the negatively charged GAG on the cell surface and HBP's strongly positively charged surface area, and another between the positively charged domain 5 of HMWK and HBP's negatively charged surface area.

Further, in addition to the above-mentioned highly charged binding sites HBP also carries other putative binding sites, such as binding sites for the Lipid A part in LPS and for interaction with and activation of Protein Kinase C (PKC).

Antibodies directed to different epitopes on HBP are of considerable pharmaceutical interest as drug candidates for the prevention and/or the treatment of infections, local and systemic inflammatory disorders, asthma, systemic inflammatory response syndrome (SIRS), degenerative diseases (Alzheimer's disease), pain and other serious diseases and disorders. Functional antibodies against HBP by binding to different epitopes comprised by the binding sites to putative HBP receptors, may modulate the functioning HBP in disease, improving or impairing the HBP capabilities as a mediator of the inflammatory response.

The invention features two groups of anti-HBP functional antibodies:

One group of antibodies is the antibodies that have agonistic capacities to HBP functions. These antibodies according to the invention are capable of recognition of en epitope(s) located within a fragment of HBP comprising amino acid residues 1 to 19 or 45 to 225 (according to SEQ ID NO: 1) concerns by the invention as powerful stimulators of different HBP activities involved in mediation of the inflammatory response. These antibodies may according to the invention serve as
(i) clustering agents increasing a local concentration of HBP at the sites of inflammation,
(ii) enhancers of the biological activity of 20-44 fragment of HBP, which has been shown to be of major importance for example for bactericidal function of the protein or in activation of monocytes;
(iii) co-factors that favors the interaction between HBP and a HBP receptor, when the binding of an antibody to HBP leads to a conformational chance in the structure of the protein that favors the interaction.

Another group of antibodies of interest is a group consisting of anti-HBP antibodies having antagonistic capacities to HBP function. These antibodies according to the invention are capable to recognize an epitope(s) within the sequence of HBP comprising amino acid residues 20-44 (SEQ ID NO: 1). Binding to such epitopes according to invention may lead to inhibiting the functions of HBP, such as for example monocyte activation and releasing the mediators of inflammation e.g. IL-6.

Medicament

It is an important objective of the present invention to use the antibodies, functionally active fragments or variants of said antibodies for the manufacture of a medicament for prevention and/or treatment of Gram positive and/or Gram negative infections, sepsis, severe sepsis, septic shock and/or disseminated intravascular coagulation, and/or for modulation of inflammatory response, and/or prevention of cell apoptosis.

In one embodiment the invention relates to the manufacture of a medicament which is capable of being used for prevention and/or treatment of Gram positive bacterial infection caused by Bacillaceae, Micrococcaceae, Mycobacteriaceae, Peptococcaceae and/or a Gram negative bacterial infection caused by Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae, Spirochaetaceae, Vibrionaceae, Brucella, Chromobacterium.

In a preferred embodiment for prevention and/or treatment the infection by Neisserie meningitidis(meningococcus) and/or Pneumococcus pneumonae (pneumococcus).

In another embodiment the invention relates to the manufacture of a medicament which may be used for prevention and/or treatment of sepsis, severe sepsis, septic shock and disseminated intravascular coagulation.

It is an important objective of the invention to use the antibodies for the manufacture of a medicament for stimulation of an inflammatory response, in a preferred embodiment, the inflammatory response to bacterial infection.

Another important objective of the invention is to use the antibodies for the manufacture of a medicament for inhibition of an inflammatory response. Examples of inflammatory responses, which may be harmful for an individual and therefore are advantageously being suppressed include but are not limited by conditions associated with extensive trauma, or chronic inflammation, such as for example type IV delayed hypersensitivity, associated for example with infection by Tubercle bacilli, or systematic inflammatory response syndrome, or multiple organ failure, or rheumatoid arthritis.

Specifically, antibody F19A5B1, which is capable of binding to an epitope comprising one or more amino acid residues of a sequence comprising amino acid residues 1 to 19 or 45 to 225 of the sequence set forth in SEQ ID NO: 1 and thereby stimulating at least one inflammatory response associated with one or more biological activities of hHBP or a hHBP fragment comprising amino acids 20-44 according to the sequence identified as SEQ ID NO: 1, preferably stimulating the production of IL-6, may be used for the manufacture of a medicament for stimulation of an inflammatory response, in a preferred embodiment, the inflammatory response to bacterial infection. Accordingly, antibody F19A5B4, which is capable of binding an epitope comprising one or more amino acid residues of a sequence comprising amino acid residues 20 to 44 of the sequence set forth in SEQ ID NO: 1 and thereby inhibiting the inflammatory response associated with one or more biological activities of hHBP or a hHBP fragment comprising amino acids 20-44 according to the sequence identified as SEQ ID NO: 1, preferably inhibiting the production of IL-6, may be used for the manufacture of a medicament for a pathological condition wherein inhibition of inflammatory response is required. Examples of inflammatory responses, when a medicament comprising the inhibitory antibody may be used include but are not limited by the conditions associated with extensive trauma, or chronic inflammation, such as for example type IV delayed hypersensitivity, associated for example with infection by Tubercle bacilli, or systematic inflammatory response syndrome, or multiple organ failure, or rheumatoid arthritis.

In an additional embodiment the invention intend to use the above described anti-body for stimulating or inhibiting the ant-apoptotic activity of hHBP. Such antibodies may be used for the manufacture of a medicament for treatment of a disease or pathological conditions associated with massive cell loss due to apoptosis. Examples of such a disease include but not limited by degenerative diseases the central and peripheral nervous system, such as postoperative nerve damage, traumatic nerve damage, e.g. resulting from spinal cord injury, impaired myelination of nerve fibers, postischaemic damage, e.g. resulting from a stroke, multiinfarct dementia, multiple sclerosis, nerve degeneration associated with diabetes mellitus, neuromuscular degeneration, schizophrenia, Alzheimer's disease, Parkinson's disease, or Huntington's disease, degenerative conditions of the gonads, of the pancreas, such as diabetes mellitus type I and II, of the kidney, such as nephrosis, or cancer.

By the term “apoptosis” in the present content is meant a programmed cell death due to activation an internal death program.

In the pharmaceutical composition of a medicament according to the invention, the antibodies may be formulated by any of the established methods of formulating pharmaceutical compositions, e.g. as described in Remington's Pharmaceutical Sciences, 1985. The composition may typically be in a form suited for local or systemic injection or infusion and may, as such, be formulated with sterile water or an isotonic saline or glucose solution. The compositions may be sterilised by conventional sterilisation techniques, which are well known in the art. The resulting aqueous solutions may be packaged for use or filtered under aseptic conditions and lyophilised, the lyophilised preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc. The concentration of peptides may vary widely, i.e. from less than about 0.5%, such as from 1%, to as much as 15-20% by weight. A unit dosage of the composition may typically contain from about 10 mg to about 1 g of a peptide.

The antibodies may be administered topically or by injection. Dosages will be prescribed by the physician according to the particular condition and the particular individual to be treated. Dosages and frequency is carefully adapted and adjusted according to parameters determined by the physician in charge. A preferred administration route may be e.g. subcutaneous injections. Subcutaneous: intravenous, intramuscular, intratracheal, intravesical, intratechal or intraperitoneal injections of anti-HBP antibodies may be given per 24 hours in the range of from 0.1-100 mg, especially 0.1-20 mg, in particular 0.1-10 mg per kg body weight. The dose may be given 1-4 times per 24 hours or administered continuously through a catheter.

Compositions of a medicament used in the present invention comprising bioactive anti-HBP antibodies described above may additionally be supplemented by antibiotics, wherein said antibiotics are routinely prescribed antibiotics by the physician according to the particular condition and the particular individual to be treated. In a preferred embodiment the supplemented antibiotics are selected from but not limited by the group of beta-lactam antibiotics, comprising penicillins and cephalosporins. A medicament comprising an anti-HBP antibody may still additionally be supplemented by an pro-inflammatory drug, or an anti-inflammatory drug, wherein said drugs are prescribed by the physician according to the particular condition and the particular individual to be treated. The supplementary pro-inflammatory drugs may for example be selected from the group comprising CSF (colony stimulating factor) drugs. The supplementary anti-inflammatory drugs may for example be selected from the group comprising antibiotics, steroids, cytostatics, or antiviral drugs.

### EXPERIMENTALS

### Example 1

### Production of Monoclonal Antibodies

Methods

Production of Monoclonal Antibodies

A panel of monoclonal antibodies against pHBP were produced according to method of Kohler G. and Milstein C (Nature 1975; 256:495-497). The antibodies were purified using ImmunoPure® IgG (Protein A) Purification Kit (PIERCE) and tested in the whole blood assay as described below.

Purified antibodies have been screened for a capability to inhibit or stimulate production of IL-6 in whole human blood in the presence or absence of h20-44 peptide alone or in the presence of h20-44 peptide and PGN or PCW from Staphilococcus aureus.

Whole Blood Screening Assay

Human whole blood (WB) samples contain besides red cells, platelets and plasma the white blood cells including the neutrophils and monocytes. Neutrophils and monocytes have receptors for bacterial products such as LPS, PGN and LTA. The bacterial products react directly or via specific binding proteins to receptors on the monocytes thereby stimulating them to secrete and release inflammatory cytokines such as IL-1, IL-6, and TNF-α. HBP and HBP fragments, specifically the fragments comprising 20-44 aa according to SEQ ID NO: 1 or 2 do not influence the production of cytokines when they present in the blood, however they can significantly amplify cytokine synthesis and secretion induced by bacterial products. In the assay the amplification of 160 μM HBP per ml WB typically leads to at least three-fold amplification of the cytokine secretion.

In the screening assay LPS from the E. coli was used to stimulate the monocytes in WB (anti-coagulated by use of citrate) to secrete IL-6 and activity of the antibodies was evaluated in relation to the amount of IL-6 in plasma subsequently separated from WB. The activity was related to the capability to:
5. increase IL-6 secretion in absence of bacterial products
6. increase IL-6 secretion in presence of bacterial products
7. decrease IL-6 secretion in presence of bacterial products
8. inhibit amplification of IL-6 secretion induced by intact HBP
   Reagents and Methods

All operations must be carried out in LAF cabinet by observance of stringent aseptic techniques. All test tubes, pipette tips etc. must be pyrogen-free. Buffers must be prepared by use of sterile, pyrogen water, preferably water for injection. Use 0.1% pyrogen-free BSA/PBS for all dilutions.

Add 20 μl of the antibody (in concentrations from 25 to 2500 μg/ml) to 100 μl freshly drawn (less than 4 hours old) citrate whole blood from a healthy human volunteer. Add 20 μl bacterial component (LPS, LTA or PGN) in concentrations from 5 to 5000 ng/ml, preferably 50 to 500 ng/ml. Mix well and incubate for 16-18 hours in an atmosphere of 5% carbon dioxide and at least 95% relative humidity. At the end of the incubation add at least 5 volumes (700 μl) 0.1% BSA/PBS. Mix well. Centrifuge 10 min. at 10.000 g. Aspirate 500 μl supernatant. Determine the level of IL-6 by a specific human immune assay for human IL-6 with sensitivity of at least 3 pg/ml, e.g. Human IL-6 Kit from RnD Systems (cat. no. D 6050).

Negative controls: 100 μl WB plus 40 μl 0.1% BSA/PBS. Positive control: 100 μl WB plus 20 μl LPS (same concentration as used for testing the peptide) and 20 μl 0.1% BSA/PBS.

Results

TABLE 1

| Blood | F19A5B1 | F19A5B4 | pHBP | 0.1% BSA | PCW | IL-6 |
|---|---|---|---|---|---|---|
| μl | mg/ml | μg/ml | mg/ml | μl | μg/ml | pg/ml |
| 100 | | | | 60 | | 10.4 |
| 100 | | | | 60 | | 13.0 |
| 100 | 168 | | | 40 | | 3213.6 |
| 100 | 168 | | | 40 | | 3857.1 |
| 100 | | 168 | | 40 | | 10.4 |
| 100 | | 168 | | 40 | | 14.3 |
| 100 | 168 | | 25 | 20 | 90 | 5255.9 |
| 100 | 168 | | 25 | | 90 | 4755.4 |
| 100 | | 168 | 25 | 20 | 90 | 6.5 |
| 100 | | 168 | | 20 | 90 | 1.4 |

From table 1 it appears that the monoclonal antibody produced by clone F19A5B1 (fusion No 19 well A5 sub-cloned and well B1 selected) enhanced the IL-6 secretion when added alone, and also stimulated the IL-6 production when added together with PCW and pHBP in the whole blood assay described above. From the table it also appears that the monoclonal antibody F19A5B4, obtained from well 4 in the same sub-cloning of F19A5, had the opposite effect resulting in inhibition of the IL-6 production when added either alone of in the presence of PCW and pHBP.

TABLE 2

| | hHBP 20-44 | | | | |
|---|---|---|---|---|---|
| Blood | peptide | F19A5B4 | | PGN | IL-6 |
| ml | μg/ml | mg/ml | 0.1% BSA | mg/ml | pg/ml |
| 100 | 360 | | 40 | | 510.5 |
| 100 | 360 | | 40 | | 366.6 |
| 100 | 180 | | 40 | | 122.7 |
| 100 | 180 | | 40 | | 142.7 |
| 100 | 90 | | 40 | | 60.7 |
| 100 | 90 | | 40 | | 51.9 |
| 100 | | | 40 | 250 | 15331.1 |
| 100 | | | 40 | 250 | 13856.5 |
| 100 | 360 | | 20 | 250 | 27639.4 |
| 100 | 360 | | 20 | 250 | 23178.0 |
| 100 | 180 | | 20 | 250 | 19289.1 |
| 100 | 180 | | 20 | 250 | 22415-3 |
| 100 | 90 | | 20 | 250 | 14668.9 |
| 100 | 90 | | 20 | 250 | 8109.7 |
| 100 | 45 | | 20 | 250 | 10619.0 |
| 100 | 45 | | 20 | 250 | 9783.6 |
| 100 | 360 | 168 | 20 | | 317.4 |
| 100 | 360 | 168 | 20 | | 255.7 |
| 100 | 180 | 168 | 20 | | 80.7 |
| 100 | 180 | 168 | 20 | | 70.6 |
| 100 | 90 | 168 | 20 | | 56.8 |
| 100 | 90 | 168 | 20 | | 60.7 |
| 100 | 45 | 168 | 20 | | 17.3 |
| 100 | 45 | 168 | 20 | | 16.4 |
| 100 | 360 | 168 | | 250 | 3638.7 |
| 100 | 360 | 168 | | 250 | 2615.7 |
| 100 | 180 | 168 | | 250 | 730.1 |
| 100 | 180 | 168 | | 250 | 1006.4 |
| 100 | 90 | 168 | | 250 | 337.7 |
| 100 | 90 | 168 | | 250 | 145.0 |
| 100 | 45 | 168 | | 250 | 60.1 |

From table 2 it appears that human 20-44 peptide acetylated in the N terminal and amidated in the C terminal respectively enhanced the IL-6 secretion when added alone and also enhanced the IL-6 secretion when added together with PGN. However, the monoclonal antibody F19A5B4 inhibits these effects of the peptide.

Conclusion

Highly specific monoclonal and also polyclonal antibodies raised against HBP or peptides thereof can be produced and selected on their capability to stimulate the immune response (measured as IL-6 secretion but not restricted to this cytokine as a number of other cytokines such as TNF-alpha, IL-1, IL-8 can also be measured in the same time). Thus, compounds comprising such antibodies would potentiate the immune response induces by bacterial products

Highly specific monoclonal and also polyclonal antibodies raised against HBP or peptide fragments thereof can be produced and selected for their capability to inhibit the immune response (measured as IL-6 secretion but not restricted to this cytokine as a number of other cytokines such as TNT-alpha, IL-1, IL-6 can be measured in the same time). Such antibodies will inhibit the immune response induced by bacterial products and/or the immune response induces by endogenous HBP or peptide fragments thereof added exogenously, specifically the response induced by the fragment of human HBP consisting amino acids 20-44 according to the sequence identified as SEQ ID NO: 1, wherein the C-terminus of said fragment is acetylated and the N-terminus is amidated. Thus, a compound comprising such antibodies would inhibit the amplification of the immune response induces by the endogenous HBP or HBP peptide fragments added exogenously in the presence of bacterial lipopolysaccarides in the blood.

### Example 2

### Reduction of Toxic Shock Syndrome Lung Damage by Anti-HBP Antibody

Background

Streptococcus pyogenes is a major human bacterial pathogen, which frequently causes a severe illness known as hyperacute toxic shock syndrome (TSS). TSS is characterized by extensive plasma leakage, circulatory shock, and fibrin deposits into the lungs causing respiratory failure with high mortality rate. During an infection Streptococcus pyogenes expresses and releases substantial amounts of the M protein, which is an exotoxin responsible for the virulence of this bacteria and the major pathogenic factor in TSS.

Mouse Model of Severe Toxic Shock Syndrome

Dr. Heiko Herwald and colleagues at Lund University, Sweden, have developed a model for provocation of TSS in mice. In this model, M protein is injected intravenously (i.v.) into mice. Within 30 minutes the mice develop signs of severe shock. The mice are sacrificed after 30 minutes and the lungs are examined by scanning electron microscopy, which show evidence of severe destruction of the normal alveolar structure and extensive fibrin deposits, which can be seen as evidence of extensive plasma leakage. HBP, a central pro-inflammatory mediator, plays a significant pathogenic role in the toxic shock syndrome caused by the Streptococcus pyogenes M protein. Anti-human HBP antibody significantly reduces lung damage. Mice have an HBP-like molecule, which is capable to reacts with certain antibodies raised against human HBP. In vitro studies have shown that such antibodies significantly inhibit the inflammatory reactions caused by the M protein. It was therefore evaluated if such antibodies could also prevent or reduce the lung damage caused by i.v. injection of the M protein in mice. Adult mice were divided into three groups, 3 mice in each group. The first group served as control and received only PBS. The second group received 15 μg M protein intravenously and the third group received 15 μg M protein plus 100 microgram of a polyclonal rabbit anti-human HBP antibody previously shown to cross-react with the putative mouse HBP molecule. Thirty minutes after the injection the mice were sacrificed and the lungs removed.

Results

The lungs were examined by scanning electron microscopy. The control group as expected showed normal lung structure. In the group given the M protein, severe lung damage was observed: the alveolar structure was destroyed, the remaining walls were thickened and the alveoli were filled up with deposits. In the group given M protein and anti-HBP antibody it appeared that between ⅔ and ¾ of the alveoli and normal alveolar structure were preserved, indicating a dramatic reduction in the lung damage. Careful examination of all the electron microscopy pictures taken revealed the same observations in all three mice within each group.

Discussion and Conclusion

Human HBP has been shown to be responsible for neutrophil evoked vascular leakage in an in vitro model (Gautam et al. Nature Medicine 2001; 7:1-5). Anti-HBP anti-bodies might be an interesting approach to development of a new class of anti-inflammatory drugs. The results presented provide the first in vivo evidence that anti-HBP antibodies may actually be therapeutically useful in prevention of severe inflammatory lung damage.

1. A pharmaceutical composition for modulating at least one inflammatory response associated with human heparin binding protein (hHBP), said composition comprising an antibody against hHBP (SEQ ID NO: 1) or a fragment of said antibody, or an antibody against a homologue of hHBP or a fragment of said antibody, wherein the antibody (i) is capable of binding to an epitope within the sequence consisting of amino acid residues 1 to 19 or 45 to 226 according to SEQ ID NO: 1 and thereby stimulating at least one inflammatory response associated with hHBP, or (ii) is capable of binding to an epitope within the sequence consisting of amino acid residues 20 to 44 according to SEQ ID NO: 1 and thereby inhibiting at least one inflammatory response associated with hHBP.

2. The pharmaceutical composition according to claim 1, wherein the composition is for the stimulating at least one inflammatory response associated with human heparin binding protein (hHBP), said composition comprising an antibody against hHBP (SEQ ID NO: 1) or a fragment of said antibody, or an antibody against a homologue of hHBP or a fragment of said antibody, said antibody being capable of binding to an epitope within the sequence consisting of amino acid residues 1 to 19 or 45 to 226 according to SEQ ID NO: 1 and thereby stimulating at least one inflammatory response associated with hHBP.

3. The pharmaceutical composition according to claim 1, wherein the composition is for the inhibiting at least one inflammatory response associated with human heparin binding protein (hHBP), said composition comprising an antibody against hHBP (SEQ ID NO: 1) or a fragment of said antibody, or an antibody against a homologue of hHBP or a fragment of said antibody, said antibody being capable of binding to an epitope within the sequence comprising amino acid residues 20 to 44 according to SEQ ID NO: 1 and thereby inhibiting at least one inflammatory response associated with hHBP.

4. The pharmaceutical composition according to claim 1, wherein the antibody is a monoclonal antibody.

5. The pharmaceutical composition according to claim 1, wherein the antibody is produced by a cell of clone F19A5B1 (ECACC Ass. No.: 03090301)

6. The pharmaceutical composition according to claim 1, wherein the antibody is produced by a cell of clone F19A5B4 (ECACC Ass. No.: 03090302).

7. The pharmaceutical composition according to claim 1, wherein the antibody is a polyclonal antibody.

8. The pharmaceutical composition according to claim 1, wherein the HBP homologue is porcine heparin binding protein (pHBP) (SEQ ID NO: 588).

9. The pharmaceutical composition according to claim 1, wherein the HBP homologue is human neutrophil elastase (hNEL) (SEQ ID NO: 589).

10. The pharmaceutical composition according to claim 1, wherein the modulating of at least one inflammatory response being
i) up- or down regulating the gene expression in the immune cells, preferably monocytes/macrophages, leading to secretion of endogenous inflammatory mediators including receptors for inflammatory mediators and transcription factors involved in the signal tranduction of the inflammatory mediators, activation of the production of bradykinin by the phase contact system, and/or
ii) increasing or decreasing the blood concentration of monocytes and/or local accumulation thereof at the sites of inflammation, and/or
iii) increasing or decreasing the life-time of monocytes, neutrophils and other immune cells due to inhibition of apoptosis, and/or
iv) activating or inhibiting the expression of adhesion molecules by the vascular endothelial cells, and/or
v) activating or inhibiting the contact phase system producing bradykinin leading to an increased vascular permeability, and/or
vi) increasing the phagocytic potential of monocytes/macrophages, and/or
vii) up-regulation of class-II MHC.

11. The pharmaceutical composition according to claim 10, wherein the immune cells are monocytes/macrophages.

12. The pharmaceutical composition according to claim 10, wherein the mediators are cytokines, selected from the group consisting of TNFalpha, IL-1, IL-6, G-CSF, GM-CSF, and M-CSF, chemokines selected from the group consisting of IL-8 and MCP-1, or receptors selected from the group consisting of Tissue factor and IL-2Ralpha.

13. The pharmaceutical composition according to claim 10, wherein the adhesion molecules are selected from the group comprising PECAM, ICAM-1, E-selectins and VCAM-1.

14. The pharmaceutical composition according to claim 10, wherein the antibody is a pro-inflammatory antibody capable of stimulating the at least one such inflammatory response in the absence of bacterial products in the blood.

15. The pharmaceutical composition according to claim 10 wherein the antibody is a pro-inflammatory antibody capable of stimulating the at least one such inflammatory response in synergistic action with bacterial products present in the blood.

16. The pharmaceutical composition according to claim 10, wherein the antibody is capable of stimulating the synthesis and/or release of cytokine IL-6.

17. (canceled)

18. The pharmaceutical composition according to claim 10, wherein the antibody is an anti-inflammatory antibody capable of inhibiting the at least one such inflammatory response in the absence of bacterial products in the blood.

19. The pharmaceutical composition according to claim 10, wherein the antibody is an anti-inflammatory antibody capable of inhibiting the at least one such inflammatory response as defined in claims 10-13 in the presence of bacterial products in the blood.

20. (canceled)

21. The pharmaceutical composition according to claim 15, wherein the bacterial products are selected from the group consisting of LPS (Lipopolysaccharide), PGN (peptidoglycan), LTA (Lipotechoic acid), MDP (muramyldipeptide) and PCW (purified cell wall from bacteria).

22. The pharmaceutical composition according to claim 10 wherein the antibody fragment is capable of binding to
(i) an epitope within the sequence consisting of amino acid residues 1 to 19 or 45 to 226 according to SEQ ID NO: 1 and thereby activating at least one such inflammatory response, or
(ii) an epitope within the sequence consisting of amino acid residues 20-44 according to SEQ ID NO: 1 and thereby inhibiting at least one such inflammatory response.

23. An hHPB binding pro-inflammatory monoclonal antibody having all of the identifying characteristics of the monoclonal antibody produced by clone F19A5B1 (ECACC 03090301), or an antibody fragment which is an hHBP-binding fragment of said monoclonal antibody.

24. An hHBP binding anti-inflammatory monoclonal antibody having all of the identifying characteristics of the monoclonal antibody produced by clone F19A5B4 (ECACC 03090302), or an antibody fragment which is an hHBP-binding fragment of said monoclonal antibody.

25. A cell producing the antibody according to claim 21.

26. A cell producing the antibody according to claim 22.

27. An antibody or fragment thereof, wherein said antibody or said fragment is capable of binding to an epitope in hHBP, wherein said epitope being an epitope as according to claim 1.

28. A recombinant protein comprising the antibody fragment of the antibody of claim 23, said fragment being capable of binding to an epitope within the sequence consisting of amino acid residues 1 to 19 or 45 to 226 according to SEQ ID NO: 1 and thereby activating at least one inflammatory response.

29. A recombinant protein comprising the antibody fragment of the antibody of claim 23, said fragment being capable of binding to an epitope within the sequence consisting of amino acid residues 20 to 44 according to SEQ ID NO: 1 and thereby inhibiting at least one inflammatory response.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A method of modulating the inflammatory response in a subject which comprises administering an inflammatory response-modulating amount of a composition according to claim 1.

45. A method of stimulating the inflammatory response in a subject which comprises administering an inflammatory response-stimulating amount of a composition according to claim 2.

46. A method of inhibiting the inflammatory response in a subject which comprises administering an inflammatory response-inhibiting amount of a composition according to claim 3.

47. The method of claim 45, wherein said composition comprises antibody F19A5B1 or a hHBP-binding fragment thereof.

48. The method of claim 46, wherein said composition comprises antibody F19A5B4 or a hHBP-binding fragment thereof.

49. The method of claim 44, wherein the inflammatory response is a response to bacterial infection.

50. The method of claim 49, wherein the infection is a Gram negative bacterial infection.

51. The method of claim 49, wherein the infection is a Gram positive bacterial infection.

52. The method of claim 46, wherein the inflammatory response is associated with sepsis, severe sepsis, sepsis shock and/or disseminated intravascular coagulation.

53. The method of claim 44, wherein the inflammatory response is associated with meningitis.

54. The method of claim 53, wherein the meningitis is meningococcal meningitis.

55. The method according to claim 51, wherein the infection is by Pneumococcus pneumoniae.

56. A method for treating individuals having suppressed immune system, cancer, autoimmune diseases and/or trauma comprising administering an effective amount of antibody F19A5B1.

57. A method for treating individuals having a sustained inflammatory response comprising administering an effective amount of antibody F19A5B4.


SEQUENCE LISTING (589 sequences)

SEQ ID NO: 1 - protein, 225 aa, Homo sapiens
IVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 2 - protein, 4 aa, Artificial sequence
  peptide fragment amino acid residues 7-10 of hHBP
ARPR

SEQ ID NO: 3 - protein, 4 aa, Artificial sequence
  peptide fragment amino acid residues 8-11 of hHBP
RPRQ

SEQ ID NO: 4 - protein, 4 aa, Artificial sequence
  peptide fragment amino acid residues 9-12 of hHBP
PRQF

SEQ ID NO: 5 - protein, 4 aa, Artificial sequence
  peptide fragment amino acid residues 10-13 of hHBP
RQFQ

SEQ ID NO: 6 - protein, 4 aa, Artificial sequence
  peptide fragment amino acid residues 11-14 of hHBP
QFQF

SEQ ID NO: 7 - protein, 4 aa, Artificial sequence
  peptide fragment amino acid residues 12-15 of hHBP
FQFL

SEQ ID NO: 8 - protein, 4 aa, Artificial sequence
  peptide fragment amino acid residues 13-16 of hHBP
QFLA

SEQ ID NO: 9 - protein, 4 aa, Artificial sequence
  peptide fragment amino acid residues 14-17 of hHBP
FLAS

SEQ ID NO: 10 - protein, 4 aa, Artificial sequence
  peptide fragment amino acid residues 15-18 of hHBP
LASI

SEQ ID NO: 11 - protein, 4 aa, Artificial sequence
  peptide fragment amino acid residues 16-19 of hHBP
ASIQ

SEQ ID NO: 12 - protein, 4 aa, Artificial sequence
  peptide fragment amino acid residues 17-20 of hHBP
SIQN

SEQ ID NO: 13 - protein, 4 aa, Artificial sequence
  peptide fragment amino acid residues 18-21 of hHBP
IQNQ

SEQ ID NO: 14 - protein, 4 aa, Artificial sequence
  peptide fragment amino acid residues 19-22 of hHBP
QNQG

SEQ ID NO: 15 - protein, 4 aa, Artificial sequence
  peptide fragment amino acid residues 20-23 of hHBP
NQGR

SEQ ID NO: 16 - protein, 4 aa, Artificial sequence
  peptide fragment amino acid residues 21-24 of hHBP
QGRH

SEQ ID NO: 17 - protein, 4 aa, Artificial sequence
  peptide fragment amino acid residues 22-25 of hHBP
GRHF

SEQ ID NO: 18 - protein, 4 aa, Artificial sequence
  peptide fragment amino acid residues 23-26 of hHBP
RHFC

SEQ ID NO: 19 - protein, 4 aa, Artificial sequence
  peptide fragment amino acid residues 24-27 of hHBP
HFCG

SEQ ID NO: 20 - protein, 4 aa, Artificial sequence
  peptide fragment amino acid residues 25-28 of hHBP
FCGG

SEQ ID NO: 21 - protein, 4 aa, Artificial sequence
  peptide fragment amino acid residues 26-29 of hHBP
CGGA

SEQ ID NO: 22 - protein, 4 aa, Artificial sequence
  peptide fragment amino acid residues 27-30 of hHBP
GGAL

SEQ ID NO: 23 - protein, 4 aa, Artificial sequence
  peptide fragment amino acid residues 28-31 of hHBP
GALI

SEQ ID NO: 24 - protein, 4 aa, Artificial sequence
  peptide fragment amino acid residues 29-32 of hHBP
ALIH

SEQ ID NO: 25 - protein, 4 aa, Artificial sequence
  peptide fragment amino acid residues 30-33 of hHBP
LIHA

SEQ ID NO: 26 - protein, 4 aa, Artificial sequence
  peptide fragment amino acid residues 31-34 of hHBP
IHAR

SEQ ID NO: 27 - protein, 4 aa, Artificial sequence
  peptide fragment amino acid residues 32-35 of hHBP
HARF

SEQ ID NO: 28 - protein, 4 aa, Artificial sequence
  Peptide fragment amino acid residues 33-36 of hHBP
ARFV

SEQ ID NO: 29 - protein, 4 aa, Artificial sequence
  Peptide fragment amino acid residues 34-37 of hHBP
RFVM

SEQ ID NO: 30 - protein, 4 aa, Artificial sequence
  Peptide fragment amino acid residues 35-38 of hHBP
FVMT

SEQ ID NO: 31 - protein, 4 aa, Artificial sequence
  Peptide fragment amino acid residues 36-39 of hHBP
VMTA

SEQ ID NO: 32 - protein, 4 aa, Artificial sequence
  Peptide fragment amino acid residues 37-40 of hHBP
MTAA

SEQ ID NO: 33 - protein, 4 aa, Artificial sequence
  Peptide fragment amino acid residues 38-41 of hHBP
TAAS

SEQ ID NO: 34 - protein, 4 aa, Artificial sequence
  Peptide fragment amino acid residues 39-42 of hHBP
AASC

SEQ ID NO: 35 - protein, 4 aa, Artificial sequence
  Peptide fragment amino acid residues 40-43 of hHBP
ASCF

SEQ ID NO: 36 - protein, 4 aa, Artificial sequence
  Peptide fragment amino acid residues 41-44 of hHBP
SCFQ

SEQ ID NO: 37 - protein, 4 aa, Artificial sequence
  Peptide fragment amino acid residues 42-45 of hHBP
CFQS

SEQ ID NO: 38 - protein, 4 aa, Artificial sequence
  Peptide fragment amino acid residues 43-46 of hHBP
FQSQ

SEQ ID NO: 39 - protein, 4 aa, Artificial sequence
  Peptide fragment amino acid residues44-47 of hHBP
QSQN

SEQ ID NO: 40 - protein, 4 aa, Artificial sequence
  Peptide fragment amino acid residues 45-48 of hHBP
SQNP

SEQ ID NO: 41 - protein, 4 aa, Artificial sequence
  Peptide fragment amino acid residues46-49 of hHBP
QNPG

SEQ ID NO: 42 - protein, 4 aa, Artificial sequence
  Peptide fragment amino acid residues 47-50 of hHBP
NPGV

SEQ ID NO: 43 - protein, 4 aa, Artificial sequence
  Peptide fragment amino acid residues 48-51 of hHBP
PGVS

SEQ ID NO: 44 - protein, 4 aa, Artificial sequence
  Peptide fragment amino acid residues 49-52 of hHBP
GVST

SEQ ID NO: 45 - protein, 4 aa, Artificial sequence
  Peptide fragment amino acid residues 50-53 of hHBP
VSTV

SEQ ID NO: 46 - protein, 4 aa, Artificial sequence
  Peptide fragment amino acid residues 51-54 of hHBP
STVV

SEQ ID NO: 47 - protein, 4 aa, Artificial sequence
  Peptide fragment amino acid residues 52-55 of hHBP
TVVL

SEQ ID NO: 48 - protein, 4 aa, Artificial sequence
  Peptide fragment amino acid residues 53-56 of hHBP
VVLG

SEQ ID NO: 49 - protein, 4 aa, Artificial sequence
  Peptide fragment amino acid residues 54-57 of hHBP
VLGA

SEQ ID NO: 50 - protein, 4 aa, Artificial sequence
  Peptide fragment amino acid residues 55-58 of hHBP
LGAY

SEQ ID NO: 51 - protein, 4 aa, Artificial sequence
  Peptide fragment amino acid residues 56-59 of hHBP
GAYD

SEQ ID NO: 52 - protein, 4 aa, Artificial sequence
  Peptide fragment amino acid residues 57-60 of hHBP
AYDL

SEQ ID NO: 53 - protein, 4 aa, Artificial sequence
  Peptide fragment amino acid residues 58-61 of hHBP
YDLR

SEQ ID NO: 54 - protein, 4 aa, Artificial sequence
  Peptide fragment amino acid residues 59-62 of hHBP
DLRR

SEQ ID NO: 55 - protein, 4 aa, Artificial sequence
  Peptide fragment amino acid residues 60-63 of hHBP
LRRR

SEQ ID NO: 56 - protein, 4 aa, Artificial sequence
  Peptide fragment amino acid residues 61-62 of hHBP
RRRE

SEQ ID NO: 57 - protein, 4 aa, Artificial sequence
  Peptide fragment amino acid residues 62-65 of hHBP
RRER

SEQ ID NO: 58 - protein, 4 aa, Artificial sequence
  Peptide fragment amino acid residues 63-66 of hHBP
RERQ

SEQ ID NO: 59 - protein, 4 aa, Artificial sequence
  Peptide fragment amino acid residues 64-67 of hHBP
ERQS

SEQ ID NO: 60 - protein, 4 aa, Artificial sequence
  Peptide fragment amino acid residues 65-68 of hHBP
RQSR

SEQ ID NO: 61 - protein, 4 aa, Artificial sequence
  Peptide fragment amino acid residues 66-69 of hHBP
QSRQ

SEQ ID NO: 62 - protein, 4 aa, Artificial sequence
  Peptide fragment amino acid residues 67-70 of hHBP
SRQT

SEQ ID NO: 63 - protein, 4 aa, Artificial sequence
  Peptide fragment amino acid residues 68-71 of hHBP
RQTF

SEQ ID NO: 64 - protein, 4 aa, Artificial sequence
  Peptide fragment amino acid residues 69-72 of hHBP
QTFS

SEQ ID NO: 65 - protein, 4 aa, Artificial sequence
  Peptide fragment amino acid residues 70-73 of hHBP
TFSI

SEQ ID NO: 66 - protein, 4 aa, Artificial sequence
  Peptide fragment amino acid residues 71-74 of hHBP
FSIS

SEQ ID NO: 67 - protein, 4 aa, Artificial sequence
  Peptide fragment amino acid residues 72-75 of hHBP
SISS

SEQ ID NO: 68 - protein, 4 aa, Artificial sequence
  Peptide fragment amino acid residues 73-76 of hHBP
ISSM

SEQ ID NO: 69 - protein, 4 aa, Artificial sequence
  Peptide fragment amino acid residues 74-77 of hHBP
SSMS

SEQ ID NO: 70 - protein, 4 aa, Artificial sequence
  Peptide fragment amino acid residues 75-78 of hHBP
SMSE

SEQ ID NO: 71 - protein, 4 aa, Artificial sequence
  Peptide fragment amino acid residues 76-79 of hHBP
MSEN

SEQ ID NO: 72 - protein, 4 aa, Artificial sequence
  Peptide fragment amino acid residues 77-80 of hHBP
SENG

SEQ ID NO: 73 - protein, 4 aa, Artificial sequence
  Peptide fragment amino acid residues 78-81 of hHBP
ENGY

SEQ ID NO: 74 - protein, 4 aa, Artificial sequence
  Peptide fragment amino acid residues 79-82 of hHBP
NGYD

SEQ ID NO: 75 - protein, 4 aa, Artificial sequence
  Peptide fragment amino acid residues 80-83 of hHBP
GYDP

SEQ ID NO: 76 - protein, 4 aa, Artificial sequence
  Peptide fragment amino acid residues 81-84 of hHBP
YDPQ

SEQ ID NO: 77 - protein, 4 aa, Artificial sequence
  Peptide fragment amino acid residues 82-85 of hHBP
DPQQ

SEQ ID NO: 78 - protein, 4 aa, Artificial sequence
  Peptide fragment amino acid residues 83-86 of hHBP
PQQN

SEQ ID NO: 79 - protein, 4 aa, Artificial sequence
  Peptide fragment amino acid residues 84-87 of hHBP
QQNL

SEQ ID NO: 80 - protein, 4 aa, Artificial sequence
  Peptide fragment amino acid residues 85-88 of hHBP
QNLN

SEQ ID NO: 81 - protein, 4 aa, Artificial sequence
  Peptide fragment amino acid residues 86-89 of hHBP
NLND

SEQ ID NO: 82 - protein, 4 aa, Artificial sequence
  Peptide fragment amino acid residues 87-90 of hHBP
LNDL

SEQ ID NO: 83 - protein, 4 aa, Artificial sequence
  Peptide fragment amino acid residues 88-91 of hHBP
NDLM

SEQ ID NO: 84 - protein, 4 aa, Artificial sequence
  Peptide fragment amino acid residues 89-92 of hHBP
DLML

SEQ ID NO: 85 - protein, 4 aa, Artificial sequence
  Peptide fragment amino acid residues 90-93 of hHBP
LMLL

SEQ ID NO: 86 - protein, 4 aa, Artificial sequence
  Peptide fragment amino acid residues 91-94 of hHBP
MLLQ

SEQ ID NO: 87 - protein, 4 aa, Artificial sequence
  Peptide fragment amino acid residues 92-95 of hHBP
LLQL

SEQ ID NO: 88 - protein, 4 aa, Artificial sequence
  Peptide fragment amnino acid residues 93-96 of hHBP
LQLD

SEQ ID NO: 89 - protein, 4 aa, Artificial sequence
  Peptide fragment amnino acid residues 94-97 of hHBP
QLDR

SEQ ID NO: 90 - protein, 4 aa, Artificial sequence
  Peptide fragment amnino acid residues 95-98 of hHBP
LDRE

SEQ ID NO: 91 - protein, 4 aa, Artificial sequence
  Peptide fragment amnino acid residues 96-99 of hHBP
DREA

SEQ ID NO: 92 - protein, 4 aa, Artificial sequence
  Peptide fragment amnino acid residues 97-100 of hHBP
REAN

SEQ ID NO: 93 - protein, 4 aa, Artificial sequence
  Peptide fragment amino acid residues 98-101 of hHBP
EANL

SEQ ID NO: 94 - protein, 4 aa, Artificial sequence
  Peptide fragment amino acid residues 100-103 of hHBP
ANLT

SEQ ID NO: 95 - protein, 4 aa, Artificial sequence
  Peptide fragment amino acid residues 101-104 of hHBP
NLTS

SEQ ID NO: 96 - protein, 4 aa, Artificial sequence
  Peptide fragment amino acid residues 101-104 of hHBP
LTSS

SEQ ID NO: 97 - protein, 4 aa, Artificial sequence
  Peptide fragment amino acid residues 102-105 of hHBP
TSSV

SEQ ID NO: 98 - protein, 4 aa, Artificial sequence
  Peptide fragment amino acid residues 103-106 of hHBP
SSVT

SEQ ID NO: 99 - protein, 4 aa, Artificial sequence
  Peptide fragment amino acid residues 104-107 of hHBP
SVTI

SEQ ID NO: 100 - protein, 4 aa, Artificial sequence
  Peptide fragment amino acid residues 105-108 of hHBP
VTIL

SEQ ID NO: 101 - protein, 4 aa, Artificial sequence
  Peptide fragment amino acid residues 106-109 of hHBP
TILP

SEQ ID NO: 102 - protein, 4 aa, Artificial sequence
  Peptide fragment amino acid residues 107-110 of hHBP
ILPL

SEQ ID NO: 103 - protein, 4 aa, Artificial sequence
  Peptide fragment amino acid residues 108-111 of hHBP
LPLP

SEQ ID NO: 104 - protein, 4 aa, Artificial sequence
  Peptide fragment amino acid residues 109-112 of hHBP
PLPL

SEQ ID NO: 105 - protein, 4 aa, Artificial sequence
  Peptide fragment amino acid residues 110-113 of hHBP
LPLQ

SEQ ID NO: 106 - protein, 4 aa, Artificial sequence
  Peptide fragment amino acid residues 111-114 of hHBP
PLQN

SEQ ID NO: 107 - protein, 4 aa, Artificial sequence
  Peptide fragment amino acid residues 112-115 of hHBP
LQNA

SEQ ID NO: 108 - protein, 4 aa, Artificial sequence
  Peptide fragment amino acid residues 113-116 of hHBP
QNAT

SEQ ID NO: 109 - protein, 4 aa, Artificial sequence
  Peptide fragment amino acid residues 114-117 of hHBP
NATV

SEQ ID NO: 110 - protein, 4 aa, Artificial sequence
  Peptide fragment amino acid residues 115-118 of hHBP
ATVE

SEQ ID NO: 111 - protein, 4 aa, Artificial sequence
  Peptide fragment amino acid residues 116-119 of hHBP
TVEA

SEQ ID NO: 112 - protein, 4 aa, Artificial sequence
  Peptide fragment amino acid residues 117-120 of hHBP
VEAG

SEQ ID NO: 113 - protein, 4 aa, Artificial sequence
  Peptide fragment amino acid residues 118-121 of hHBP
EAGT

SEQ ID NO: 114 - protein, 4 aa, Artificial sequence
  Peptide fragment amino acid residues 119-122 of hHBP
AGTR

SEQ ID NO: 115 - protein, 4 aa, Artificial sequence
  Peptide fragment amino acid residues 120-123 of hHBP
GTRC

SEQ ID NO: 116 - protein, 4 aa, Artificial sequence
  Peptide fragment amino acid residues 121-124 of hHBP
TRCQ

SEQ ID NO: 117 - protein, 4 aa, Artificial sequence
  Peptide fragment amino acid residues 122-125 of hHBP
RCQV

SEQ ID NO: 118 - protein, 4 aa, Artificial sequence
  Peptide fragment amino acid residues 123-126 of hHBP
CQVA

SEQ ID NO: 119 - protein, 4 aa, Artificial sequence
  Peptide fragment amino acid residues 124-127 of hHBP
QVAG

SEQ ID NO: 120 - protein, 4 aa, Artificial sequence
  Peptide fragment amino acid residues 125-128 of hHBP
VAGW

SEQ ID NO: 121 - protein, 4 aa, Artificial sequence
  Peptide fragment amino acid residues 125-128 of hHBP
AGWG

SEQ ID NO: 122 - protein, 4 aa, Artificial sequence
  Peptide fragment amino acid residues 127-130 of hHBP
GWGS

SEQ ID NO: 123 - protein, 4 aa, Artificial sequence
  Peptide fragment amino acid residues128-131 of hHBP
WGSQ

SEQ ID NO: 124 - protein, 4 aa, Artificial sequence
  Peptide fragment amino acid residues 129-132 of hHBP
GSQR

SEQ ID NO: 125 - protein, 4 aa, Artificial sequence
  Peptide fragment amino acid residues 130-133 of hHBP
SQRS

SEQ ID NO: 126 - protein, 4 aa, Artificial sequence
  Peptide fragment amino acid residues 131-134 of hHBP
QRSG

SEQ ID NO: 127 - protein, 4 aa, Artificial sequence
  Peptide fragment amino acid residues 132-135 of hHBP
RSGG

SEQ ID NO: 128 - protein, 4 aa, Artificial sequence
  Peptide fragment amino acid residues 133-136 of hHBP
SGGR

SEQ ID NO: 129 - protein, 4 aa, Artificial sequence
  Peptide fragment amino acid residues 134-137 of hHBP
GGRL

SEQ ID NO: 130 - protein, 4 aa, Artificial sequence
  Peptide fragment amino acid residues 135-138 of hHBP
GRLS

SEQ ID NO: 131 - protein, 4 aa, Artificial sequence
  Peptide fragment amino acid residues 136-139 of hHBP
RLSR

SEQ ID NO: 132 - protein, 4 aa, Artificial sequence
  Peptide fragment amino acid residues 137-140 of hHBP
LSRF

SEQ ID NO: 133 - protein, 4 aa, Artificial sequence
  Peptide fragment amino acid residues 138-141 of hHBP
SRFP

SEQ ID NO: 134 - protein, 4 aa, Artificial sequence
  Peptide fragment amino acid residues 139-142 of hHBP
RFPR

SEQ ID NO: 135 - protein, 4 aa, Artificial sequence
  Peptide fragment amino acid residues 140-143 of hHBP
FPRF

SEQ ID NO: 136 - protein, 4 aa, Artificial sequence
  Peptide fragment amino acid residues 141-144 of hHBP
PRFV

SEQ ID NO: 137 - protein, 4 aa, Artificial sequence
  Peptide fragment amino acid residues 142-145 of hHBP
RFVN

SEQ ID NO: 138 - protein, 4 aa, Artificial sequence
  Peptide fragment amino acid residues 143-146 of hHBP
FVNV

SEQ ID NO: 139 - protein, 4 aa, Artificial sequence
  Peptide fragment amino acid residues 144-147 of hHBP
VNVT

SEQ ID NO: 140 - protein, 4 aa, Artificial sequence
  Peptide fragment amino acid residues 145-148 of hHBP
NVTV

SEQ ID NO: 141 - protein, 4 aa, Artificial sequence
  Peptide fragment amino acid residues 146-149 of hHBP
VTVT

SEQ ID NO: 142 - protein, 4 aa, Artificial sequence
  Peptide fragment amino acid residues 147-150 of hHBP
TVTP

SEQ ID NO: 143 - protein, 4 aa, Artificial sequence
  Peptide fragment amino acid residues 148-151 of hHBP
VTPE

SEQ ID NO: 144 - protein, 4 aa, Artificial sequence
  Peptide fragment amino acid residues 149-152 of hHBP
TPED

SEQ ID NO: 145 - protein, 4 aa, Artificial sequence
  Peptide fragment amino acid residues 150-153 of hHBP
PEDQ

SEQ ID NO: 146 - protein, 4 aa, Artificial sequence
  Peptide fragment amino acid residues 151-154 of hHBP
EDQC

SEQ ID NO: 147 - protein, 4 aa, Artificial sequence
  Peptide fragment amino acid residues 152-155 of hHBP
DQCR

SEQ ID NO: 148 - protein, 4 aa, Artificial sequence
  Peptide fragment amino acid residues 153-156 of hHBP
QCRP

SEQ ID NO: 149 - protein, 4 aa, Artificial sequence
  Peptide fragment amino acid residues 154-157 of hHBP
CRPN

SEQ ID NO: 150 - protein, 4 aa, Artificial sequence
  Peptide fragment amino acid residues 155-158 of hHBP
RPNN

SEQ ID NO: 151 - protein, 4 aa, Artificial sequence
  Peptide fragment amino acid residues 156-159 of hHBP
PNNV

SEQ ID NO: 152 - protein, 4 aa, Artificial sequence
  Peptide fragment amino acid residues 157-160 of hHBP
NNVC

SEQ ID NO: 153 - protein, 4 aa, Artificial sequence
  Peptide fragment amino acid residues 158-161 of hHBP
NVCT

SEQ ID NO: 154 - protein, 4 aa, Artificial sequence
  Peptide fragment amino acid residues 159-162 of hHBP
VCTG

SEQ ID NO: 155 - protein, 4 aa, Artificial sequence
  Peptide fragment amino acid residues 160-163 of hHBP
CTGV

SEQ ID NO: 156 - protein, 4 aa, Artificial sequence
  Peptide fragment amino acid residues 161-164 of hHBP
TGVL

SEQ ID NO: 157 - protein, 4 aa, Artificial sequence
  Peptide fragment amino acid residues 162-165 of hHBP
GVLT

SEQ ID NO: 158 - protein, 4 aa, Artificial sequence
  Peptide fragment amino acid residues 163-166 of hHBP
VLTR

SEQ ID NO: 159 - protein, 4 aa, Artificial sequence
  Peptide fragment amino acid residues 164-167 of hHBP
LTRR

SEQ ID NO: 160 - protein, 4 aa, Artificial sequence
  Peptide fragment amino acid residues 165-168 of hHBP
TRRG

SEQ ID NO: 161 - protein, 4 aa, Artificial sequence
  Peptide fragment amino acid residues 166-169 of hHBP
RRGG

SEQ ID NO: 162 - protein, 4 aa, Artificial sequence
  Peptide fragment amino acid residues 167-170 of hHBP
RGGI

SEQ ID NO: 163 - protein, 4 aa, Artificial sequence
  Peptide fragment amino acid residues 168-171 of hHBP
GGIC

SEQ ID NO: 164 - protein, 4 aa, Artificial sequence
  Peptide fragment amino acid residues 169-172 of hHBP
GICN

SEQ ID NO: 165 - protein, 4 aa, Artificial sequence
  Peptide fragment amino acid residues 170-173 of hHBP
ICNG

SEQ ID NO: 166 - protein, 4 aa, Artificial sequence
  Peptide fragment amino acid residues 171-174 of hHBP
CNGD

SEQ ID NO: 167 - protein, 4 aa, Artificial sequence
  Peptide fragment amino acid residues 172-175 of hHBP
NGDG

SEQ ID NO: 168 - protein, 4 aa, Artificial sequence
  Peptide fragment amino acid residues 173-176 of hHBP
GDGG

SEQ ID NO: 169 - protein, 4 aa, Artificial sequence
  Peptide fragment amino acid residues 174-177 of hHBP
DGGT

SEQ ID NO: 170 - protein, 4 aa, Artificial sequence
  Peptide fragment amino acid residues 175-178 of hHBP
GGTP

SEQ ID NO: 171 - protein, 4 aa, Artificial sequence
  Peptide fragment amino acid residues 176-179 of hHBP
GTPL

SEQ ID NO: 172 - protein, 4 aa, Artificial sequence
  Peptide fragment amino acid residues 177-180 of hHBP
TPLV

SEQ ID NO: 173 - protein, 4 aa, Artificial sequence
  Peptide fragment amino acid residues 178-181 of hHBP
PLVC

SEQ ID NO: 174 - protein, 4 aa, Artificial sequence
  Peptide fragment amino acid residues 179-181of hHBP
LVCE

SEQ ID NO: 175 - protein, 4 aa, Artificial sequence
  Peptide fragment amino acid residues 180-183 of hHBP
VCEG

SEQ ID NO: 176 - protein, 4 aa, Artificial sequence
  Peptide fragment amino acid residues 181-184 of hHBP
CEGL

SEQ ID NO: 177 - protein, 4 aa, Artificial sequence
  Peptide fragment amino acid residues 182-185 of hHBP
EGLA

SEQ ID NO: 178 - protein, 4 aa, Artificial sequence
  Peptide fragment amino acid residues 183-186 of hHBP
GLAH

SEQ ID NO: 179 - protein, 4 aa, Artificial sequence
  Peptide fragment amino acid residues 184-187 of hHBP
LAHG

SEQ ID NO: 180 - protein, 4 aa, Artificial sequence
  Peptide fragment amino acid residues 185-188 of hHBP
AHGV

SEQ ID NO: 181 - protein, 4 aa, Artificial sequence
  Peptide fragment amino acid residues 186-189 of hHBP
HGVA

SEQ ID NO: 182 - protein, 4 aa, Artificial sequence
  Peptide fragment amino acid residues 187-190 of hHBP
GVAS

SEQ ID NO: 183 - protein, 4 aa, Artificial sequence
  Peptide fragment amino acid residues 188-191 of hHBP
VASF

SEQ ID NO: 184 - protein, 4 aa, Artificial sequence
  Peptide fragment amino acid residues 189-192 of hHBP
ASFS

SEQ ID NO: 185 - protein, 4 aa, Artificial sequence
  Peptide fragment amino acid residues 190-193 of hHBP
SFSL

SEQ ID NO: 186 - protein, 4 aa, Artificial sequence
  Peptide fragment amino acid residues 191-194 of hHBP
FSLG

SEQ ID NO: 187 - protein, 4 aa, Artificial sequence
  Peptide fragment amino acid residues 192-195 of hHBP
SLGP

SEQ ID NO: 188 - protein, 4 aa, Artificial sequence
  Peptide fragment amino acid residues 193-196 of hHBP
LGPC

SEQ ID NO: 189 - protein, 4 aa, Artificial sequence
  Peptide fragment amino acid residues 194-197 of hHBP
GPCG

SEQ ID NO: 190 - protein, 4 aa, Artificial sequence
  Peptide fragment amino acid residues 195-198 of hHBP
PCGR

SEQ ID NO: 191 - protein, 4 aa, Artificial sequence
  Peptide fragment amino acid residues 196-199 of hHBP
CGRG

SEQ ID NO: 192 - protein, 4 aa, Artificial sequence
  Peptide fragment amino acid residues 197-200 of hHBP
GRGP

SEQ ID NO: 193 - protein, 4 aa, Artificial sequence
  Peptide fragment amino acid residues 198-201 of hHBP
RGPD

SEQ ID NO: 194 - protein, 4 aa, Artificial sequence
  Peptide fragment amino acid residues 199-202 of hHBP
GPDF

SEQ ID NO: 195 - protein, 4 aa, Artificial sequence
  Peptide fragment amino acid residues 200-203 of hHBP
PDFF

SEQ ID NO: 196 - protein, 4 aa, Artificial sequence
  Peptide fragment amino acid residues 201-204 of hHBP
DFFT

SEQ ID NO: 197 - protein, 4 aa, Artificial sequence
  Peptide fragment amino acid residues 202-205 of hHBP
FFTR

SEQ ID NO: 198 - protein, 4 aa, Artificial sequence
  Peptide fragment amino acid residues 203-206 of hHBP
FTRV

SEQ ID NO: 199 - protein, 4 aa, Artificial sequence
  Peptide fragment amino acid residues 204-207 of hHBP
TRVA

SEQ ID NO: 200 - protein, 4 aa, Artificial sequence
  Peptide fragment amino acid residues 205-208 of hHBP
RVAL

SEQ ID NO: 201 - protein, 4 aa, Artificial sequence
  Peptide fragment amino acid residues 206-209 of hHBP
VALF

SEQ ID NO: 202 - protein, 4 aa, Artificial sequence
  Peptide fragment amino acid residues 207-210 of hHBP
ALFR

SEQ ID NO: 203 - protein, 4 aa, Artificial sequence
  Peptide fragment amino acid residues 208-211 of hHBP
LFRD

SEQ ID NO: 204 - protein, 4 aa, Artificial sequence
  Peptide fragment amino acid residues 209-212 of hHBP
FRDW

SEQ ID NO: 205 - protein, 4 aa, Artificial sequence
  Peptide fragment amino acid residues 210-213 of hHBP
RDWI

SEQ ID NO: 206 - protein, 4 aa, Artificial sequence
  Peptide fragment amino acid residues 211-214 of hHBP
DWID

SEQ ID NO: 207 - protein, 4 aa, Artificial sequence
  Peptide fragment amino acid residues 212-215 of hHBP
WIDG

SEQ ID NO: 208 - protein, 4 aa, Artificial sequence
  Peptide fragment amino acid residues 213-216 of hHBP
IDGV

SEQ ID NO: 209 - protein, 4 aa, Artificial sequence
  Peptide fragment amino acid residues 214-217 of hHBP
DGVL

SEQ ID NO: 210 - protein, 4 aa, Artificial sequence
  Peptide fragment amino acid residues 215-218 of hHBP
GVLN

SEQ ID NO: 211 - protein, 4 aa, Artificial sequence
  Peptide fragment amino acid residues 216-219 of hHBP
VLNN

SEQ ID NO: 212 - protein, 4 aa, Artificial sequence
  Peptide fragment amino acid residues 217-220 of hHBP
LNNP

SEQ ID NO: 213 - protein, 4 aa, Artificial sequence
  Peptide fragment amino acid residues 218-221 of hHBP
NNPG

SEQ ID NO: 214 - protein, 4 aa, Artificial sequence
  Peptide fragment amino acid residues 219-222 of hHBP
NPGP

SEQ ID NO: 215 - protein, 4 aa, Artificial sequence
  Peptide fragment amino acid residues 220-223of hHBP
PGPG

SEQ ID NO: 216 - protein, 4 aa, Artificial sequence
  Peptide fragment amino acid residues 221-224 of hHBP
GPGP

SEQ ID NO: 217 - protein, 4 aa, Artificial sequence
  Peptide fragment amino acid residues 222-225 of hHBP
PGPA

SEQ ID NO: 218 - protein, 4 aa, Artificial sequence
  Peptide fragment amino acid residues 3-6 of hHBP
GGRK

SEQ ID NO: 219 - protein, 4 aa, Artificial sequence
  Peptide fragment amino acid residues 4-7 of hHBP
GRKA

SEQ ID NO: 220 - protein, 4 aa, Artificial sequence
  Peptide fragment amino acid residues 5-8 of hHBP
RKAR

SEQ ID NO: 221 - protein, 4 aa, Artificial sequence
  Peptide fragment amino acid residues  6-9 of hHBP
KARP

SEQ ID NO: 222 - protein, 4 aa, Artificial sequence
  Peptide fragment amino acid residues 3-6 of pHBP
GGRR

SEQ ID NO: 223 - protein, 4 aa, Artificial sequence
  Peptide fragment amino acid residues 4-7 of pHBP
GRRA

SEQ ID NO: 224 - protein, 4 aa, Artificial sequence
  Peptide fragment amino acid residues 5-8 of pHBP
RRAQ

SEQ ID NO: 225 - protein, 4 aa, Artificial sequence
  Peptide fragment amino acid residues 6-9 of pHBP
RAQP

SEQ ID NO: 226 - protein, 4 aa, Artificial sequence
  Peptide fragment amino acid residues 7-10 of pHBP
AQPQ

SEQ ID NO: 227 - protein, 4 aa, Artificial sequence
  Peptide fragment amino acid residues 8-11 of pHBP
QPQE

SEQ ID NO: 228 - protein, 4 aa, Artificial sequence
  Peptide fragment amino acid residues 9-12 of pHBP
PQEF

SEQ ID NO: 229 - protein, 4 aa, Artificial sequence
  Peptide fragment amino acid residues 10-13 of pHBP
QEFP

SEQ ID NO: 230 - protein, 4 aa, Artificial sequence
  Peptide fragment amino acid residues 11-14 of pHBP
EFPF

SEQ ID NO: 231 - protein, 4 aa, Artificial sequence
  Peptide fragment amino acid residues 12-15 of pHBP
FPFL

SEQ ID NO: 232 - protein, 4 aa, Artificial sequence
  Peptide fragment amino acid residues 13-16 of pHBP
PFLA

SEQ ID NO: 233 - protein, 4 aa, Artificial sequence
  Peptide fragment amino acid residues 21-24 of pHBP
QGRP

SEQ ID NO: 234 - protein, 4 aa, Artificial sequence
  Peptide fragment amino acid residues 22-25 of pHBP
GRPF

SEQ ID NO: 235 - protein, 4 aa, Artificial sequence
  Peptide fragment amino acid residues 23-26 of pHBP
RPFC

SEQ ID NO: 236 - protein, 4 aa, Artificial sequence
  Peptide fragment amino acid residues 24-27 of pHBP
PFCA

SEQ ID NO: 237 - protein, 4 aa, Artificial sequence
  Peptide fragment amino acid residues 25-28 of pHBP
FCAG

SEQ ID NO: 238 - protein, 4 aa, Artificial sequence
  Peptide fragment amino acid residues 26-29 of pHBP
CAGA

SEQ ID NO: 239 - protein, 4 aa, Artificial sequence
  Peptide fragment amino acid residues 27-30 of pHBP
AGAL

SEQ ID NO: 240 - protein, 4 aa, Artificial sequence
  Peptide fragment amino acid residues 28-31 of pHBP
GALV

SEQ ID NO: 241 - protein, 4 aa, Artificial sequence
  Peptide fragment amino acid residues 29-32 of pHBP
ALVH

SEQ ID NO: 242 - protein, 4 aa, Artificial sequence
  Peptide fragment amino acid residues 30-33 of pHBP
LVHP

SEQ ID NO: 243 - protein, 4 aa, Artificial sequence
  Peptide fragment amino acid residues 31-34 of pHBP
VHPR

SEQ ID NO: 244 - protein, 4 aa, Artificial sequence
  Peptide fragment amino acid residues 32-35 of pHBP
HPRF

SEQ ID NO: 245 - protein, 4 aa, Artificial sequence
  Peptide fragment amino acid residues 17-20 of pHBP
SIQK

SEQ ID NO: 246 - protein, 4 aa, Artificial sequence
  Peptide fragment amino acid residues 34-37 of pHBP
RFVL

SEQ ID NO: 247 - protein, 4 aa, Artificial sequence
  Peptide fragment amino acid residues 35-38 of pHBP
FVLT

SEQ ID NO: 248 - protein, 4 aa, Artificial sequence
  Peptide fragment amino acid residues 36-39 of pHBP
VLTA

SEQ ID NO: 249 - protein, 4 aa, Artificial sequence
  Peptide fragment amino acid residues 37-40 of pHBP
LTAA

SEQ ID NO: 250 - protein, 4 aa, Artificial sequence
  Peptide fragment amino acid residues 41-44 of pHBP
SCFR

SEQ ID NO: 251 - protein, 4 aa, Artificial sequence
  Peptide fragment amino acid residues 42-45 of pHBP
CFRG

SEQ ID NO: 252 - protein, 4 aa, Artificial sequence
  Peptide fragment amino acid residues 43-46 of pHBP
FRGK

SEQ ID NO: 253 - protein, 4 aa, Artificial sequence
  Peptide fragment amino acid residues 44-47 of pHBP
RGKN

SEQ ID NO: 254 - protein, 4 aa, Artificial sequence
  Peptide fragment amino acid residues 45-48 of pHBP
GKNS

SEQ ID NO: 255 - protein, 4 aa, Artificial sequence
  Peptide fragment amino acid residues 46-49 of pHBP
KNSG

SEQ ID NO: 256 - protein, 4 aa, Artificial sequence
  Peptide fragment amino acid residues 47-50 of pHBP
NSGS

SEQ ID NO: 257 - protein, 4 aa, Artificial sequence
  Peptide fragment amino acid residues 48-51 of pHBP
SGSA

SEQ ID NO: 258 - protein, 4 aa, Artificial sequence
  Peptide fragment amino acid residues 49-52 of pHBP
GSAS

SEQ ID NO: 259 - protein, 4 aa, Artificial sequence
  Peptide fragment amino acid residues 50-53 of pHBP
SASV

SEQ ID NO: 260 - protein, 4 aa, Artificial sequence
  Peptide fragment amino acid residues 51-54 of pHBP
ASVV

SEQ ID NO: 261 - protein, 4 aa, Artificial sequence
  Peptide fragment amino acid residues 52-55 of pHBP
SVVL

SEQ ID NO: 262 - protein, 4 aa, Artificial sequence
  Peptide fragment amino acid residues 59-62 of pHBP
DLRQ

SEQ ID NO: 263 - protein, 4 aa, Artificial sequence
  Peptide fragment amino acid residues 60-63 of pHBP
LRQQ

SEQ ID NO: 264 - protein, 4 aa, Artificial sequence
  Peptide fragment amino acid residues 61-64 of pHBP
RQQE

SEQ ID NO: 265 - protein, 4 aa, Artificial sequence
  Peptide fragment amino acid residues 62-65 of pHBP
QQEQ

SEQ ID NO: 266 - protein, 4 aa, Artificial sequence
  Peptide fragment amino acid residues 63-66 of pHBP
QEQS

SEQ ID NO: 267 - protein, 4 aa, Artificial sequence
  Peptide fragment amino acid residues 64-67 of pHBP
EQSR

SEQ ID NO: 268 - protein, 4 aa, Artificial sequence
  Peptide fragment amino acid residues 18-21 of pHBP
IQKQ

SEQ ID NO: 269 - protein, 4 aa, Artificial sequence
  Peptide fragment amino acid residues 70-73 of pHBP
FSIR

SEQ ID NO: 270 - protein, 4 aa, Artificial sequence
  Peptide fragment amino acid residues 71-74 of pHBP
SIRS

SEQ ID NO: 271 - protein, 4 aa, Artificial sequence
  Peptide fragment amino acid residues 72-75 of pHBP
IRSI

SEQ ID NO: 272 - protein, 4 aa, Artificial sequence
  Peptide fragment amino acid residues 73-76 of pHBP
RSIS

SEQ ID NO: 273 - protein, 4 aa, Artificial sequence
  Peptide fragment amino acid residues 74-77 of pHBP
SISQ

SEQ ID NO: 274 - protein, 4 aa, Artificial sequence
  Peptide fragment amino acid residues 75-78 of pHBP
ISQN

SEQ ID NO: 275 - protein, 4 aa, Artificial sequence
  Peptide fragment amino acid residues 76-79 of pHBP
SQNG

SEQ ID NO: 276 - protein, 4 aa, Artificial sequence
  Peptide fragment amino acid residues 77-80 of pHBP
QNGY

SEQ ID NO: 277 - protein, 4 aa, Artificial sequence
  Peptide fragment amino acid residues 80-83 of pHBP
YDPR

SEQ ID NO: 278 - protein, 4 aa, Artificial sequence
  Peptide fragment amino acid residues 81-84 of pHBP
DPRQ

SEQ ID NO: 279 - protein, 4 aa, Artificial sequence
  Peptide fragment amino acid residues 82-85 of pHBP
PRQN

SEQ ID NO: 280 - protein, 4 aa, Artificial sequence
  Peptide fragment amino acid residues 83-86 of pHBP
RQNL

SEQ ID NO: 281 - protein, 4 aa, Artificial sequence
  Peptide fragment amino acid residues 86-89 of pHBP
LNDV

SEQ ID NO: 282 - protein, 4 aa, Artificial sequence
  Peptide fragment amino acid residues 87-90 of pHBP
NDVL

SEQ ID NO: 283 - protein, 4 aa, Artificial sequence
  Peptide fragment amino acid residues 88-91 of pHBP
DVLL

SEQ ID NO: 284 - protein, 4 aa, Artificial sequence
  Peptide fragment amino acid residues 89-92 of pHBP
VLLL

SEQ ID NO: 285 - protein, 4 aa, Artificial sequence
  Peptide fragment amino acid residues 90-93 of pHBP
LLLQ

SEQ ID NO: 286 - protein, 4 aa, Artificial sequence
  Peptide fragment amino acid residues 96-99 of pHBP
REAR

SEQ ID NO: 287 - protein, 4 aa, Artificial sequence
  Peptide fragment amino acid residues 97-100 of pHBP
EARL

SEQ ID NO: 288 - protein, 4 aa, Artificial sequence
  Peptide fragment amino acid residues 98-101 of pHBP
ARLT

SEQ ID NO: 289 - protein, 4 aa, Artificial sequence
  Peptide fragment amino acid residues 99-102 of pHBP
RLTP

SEQ ID NO: 290 - protein, 4 aa, Artificial sequence
  Peptide fragment amino acid residues 100-103 of pHBP
LTPS

SEQ ID NO: 291 - protein, 4 aa, Artificial sequence
  Peptide fragment amino acid residues 101-104 of pHBP
TPSV

SEQ ID NO: 292 - protein, 4 aa, Artificial sequence
  Peptide fragment amino acid residues 102-105 of pHBP
PSVA

SEQ ID NO: 293 - protein, 4 aa, Artificial sequence
  Peptide fragment amino acid residues 103-106 of pHBP
SVAL

SEQ ID NO: 294 - protein, 4 aa, Artificial sequence
  Peptide fragment amino acid residues 104-107 of pHBP
VALV

SEQ ID NO: 295 - protein, 4 aa, Artificial sequence
  Peptide fragment amino acid residues 105-108 of pHBP
ALVP

SEQ ID NO: 296 - protein, 4 aa, Artificial sequence
  Peptide fragment amino acid residues 106-109 of pHBP
LVPL

SEQ ID NO: 297 - protein, 4 aa, Artificial sequence
  Peptide fragment amino acid residues 107-110 of pHBP
VPLP

SEQ ID NO: 298 - protein, 4 aa, Artificial sequence
  Peptide fragment amino acid residues 108-111 of pHBP
PLPP

SEQ ID NO: 299 - protein, 4 aa, Artificial sequence
  Peptide fragment amino acid residues 109-112 of pHBP
LPPQ

SEQ ID NO: 300 - protein, 4 aa, Artificial sequence
  Peptide fragment amino acid residues 110-113 of pHBP
PPQN

SEQ ID NO: 301 - protein, 4 aa, Artificial sequence
  Peptide fragment amino acid residues 111-114 of pHBP
PQNA

SEQ ID NO: 302 - protein, 4 aa, Artificial sequence
  Peptide fragment amino acid residues 112-115 of pHBP
AGTN

SEQ ID NO: 303 - protein, 4 aa, Artificial sequence
  Peptide fragment amino acid residues 113-116 of pHBP
GTNC

SEQ ID NO: 304 - protein, 4 aa, Artificial sequence
  Peptide fragment amino acid residues 114-117 of pHBP
TNCQ

SEQ ID NO: 305 - protein, 4 aa, Artificial sequence
  Peptide fragment amino acid residues 121-124 of pHBP
NCQV

SEQ ID NO: 306 - protein, 4 aa, Artificial sequence
  Peptide fragment amino acid residues 126-129 of pHBP
GWGT

SEQ ID NO: 307 - protein, 4 aa, Artificial sequence
  Peptide fragment amino acid residues 127-130 of pHBP
WGTQ

SEQ ID NO: 308 - protein, 4 aa, Artificial sequence
  Peptide fragment amino acid residues 128-131 of pHBP
GTQR

SEQ ID NO: 309 - protein, 4 aa, Artificial sequence
  Peptide fragment amino acid residues 129-132 of pHBP
TQRL

SEQ ID NO: 310 - protein, 4 aa, Artificial sequence
  Peptide fragment amino acid residues 130-133 of pHBP
QRLR

SEQ ID NO: 311 - protein, 4 aa, Artificial sequence
  Peptide fragment amino acid residues 131-134 of pHBP
RLRR

SEQ ID NO: 312 - protein, 4 aa, Artificial sequence
  Peptide fragment amino acid residues 132-135 of pHBP
LRRL

SEQ ID NO: 313 - protein, 4 aa, Artificial sequence
  Peptide fragment amino acid residues 133-136 of pHBP
RRLF

SEQ ID NO: 314 - protein, 4 aa, Artificial sequence
  Peptide fragment amino acid residues 134-137 of pHBP
RLFS

SEQ ID NO: 315 - protein, 4 aa, Artificial sequence
  Peptide fragment amino acid residues 135-138 of pHBP
LFSR

SEQ ID NO: 316 - protein, 4 aa, Artificial sequence
  Peptide fragment amino acid residues 136-139 of pHBP
FSRF

SEQ ID NO: 317 - protein, 4 aa, Artificial sequence
  Peptide fragment amino acid residues 139-142 of pHBP
FPRV

SEQ ID NO: 318 - protein, 4 aa, Artificial sequence
  Peptide fragment amino acid residues 140-143 of pHBP
PRVL

SEQ ID NO: 319 - protein, 4 aa, Artificial sequence
  Peptide fragment amino acid residues 141-144 of pHBP
RVLN

SEQ ID NO: 320 - protein, 4 aa, Artificial sequence
  Peptide fragment amino acid residues 142-145 of pHBP
VLNV

SEQ ID NO: 321 - protein, 4 aa, Artificial sequence
  Peptide fragment amino acid residues 143-146 of pHBP
LNVT

SEQ ID NO: 322 - protein, 4 aa, Artificial sequence
  Peptide fragment amino acid residues 146-149 of pHBP
TVTS

SEQ ID NO: 323 - protein, 4 aa, Artificial sequence
  Peptide fragment amino acid residues 147-150 of pHBP
VTSN

SEQ ID NO: 324 - protein, 4 aa, Artificial sequence
  Peptide fragment amino acid residues 148-151 of pHBP
TSNP

SEQ ID NO: 325 - protein, 4 aa, Artificial sequence
  Peptide fragment amino acid residues 149-152 of pHBP
SNPC

SEQ ID NO: 326 - protein, 4 aa, Artificial sequence
  Peptide fragment amino acid residues 150-153 of pHBP
NPCL

SEQ ID NO: 327 - protein, 4 aa, Artificial sequence
  Peptide fragment amino acid residues 151-154 of pHBP
PCLP

SEQ ID NO: 328 - protein, 4 aa, Artificial sequence
  Peptide fragment amino acid residues 152-155 of pHBP
CLPR

SEQ ID NO: 329 - protein, 4 aa, Artificial sequence
  Peptide fragment amino acid residues 153-156 of pHBP
LPRD

SEQ ID NO: 330 - protein, 4 aa, Artificial sequence
  Peptide fragment amino acid residues 154-157 of pHBP
PRDM

SEQ ID NO: 331 - protein, 4 aa, Artificial sequence
  Peptide fragment amino acid residues 155-158 of pHBP
RDMC

SEQ ID NO: 332 - protein, 4 aa, Artificial sequence
  Peptide fragment amino acid residues 156-159 of pHBP
DMCI

SEQ ID NO: 333 - protein, 4 aa, Artificial sequence
  Peptide fragment amino acid residues 160 of pHBP
MCIG

SEQ ID NO: 334 - protein, 4 aa, Artificial sequence
  Peptide fragment amino acid residues 158-161 of pHBP
CIGV

SEQ ID NO: 335 - protein, 4 aa, Artificial sequence
  Peptide fragment amino acid residues 159-162 of pHBP
IGVF

SEQ ID NO: 336 - protein, 4 aa, Artificial sequence
  Peptide fragment amino acid residues 160-163 of pHBP
GVFS

SEQ ID NO: 337 - protein, 4 aa, Artificial sequence
  Peptide fragment amino acid residues 161-164 of pHBP
VFSR

SEQ ID NO: 338 - protein, 4 aa, Artificial sequence
  Peptide fragment amino acid residues 162-165 of pHBP
FSRR

SEQ ID NO: 339 - protein, 4 aa, Artificial sequence
  Peptide fragment amino acid residues 163-166 of pHBP
SRRG

SEQ ID NO: 340 - protein, 4 aa, Artificial sequence
  Peptide fragment amino acid residues 164-167 of pHBP
RRGR

SEQ ID NO: 341 - protein, 4 aa, Artificial sequence
  Peptide fragment amino acid residues 165-168 of pHBP
RGRI

SEQ ID NO: 342 - protein, 4 aa, Artificial sequence
  Peptide fragment amino acid residues 166-169 of pHBP
GRIS

SEQ ID NO: 343 - protein, 4 aa, Artificial sequence
  Peptide fragment amino acid residues 167-170 of pHBP
RISQ

SEQ ID NO: 344 - protein, 4 aa, Artificial sequence
  Peptide fragment amino acid residues 168-171 of pHBP
ISQG

SEQ ID NO: 345 - protein, 4 aa, Artificial sequence
  Peptide fragment amino acid residues 169-172 of pHBP
SQGD

SEQ ID NO: 346 - protein, 4 aa, Artificial sequence
  Peptide fragment amino acid residues 170-173 of pHBP
QGDR

SEQ ID NO: 347 - protein, 4 aa, Artificial sequence
  Peptide fragment amino acid residues 171-174 of pHBP
GDRG

SEQ ID NO: 348 - protein, 4 aa, Artificial sequence
  Peptide fragment amino acid residues 172-175 of pHBP
DRGT

SEQ ID NO: 349 - protein, 4 aa, Artificial sequence
  Peptide fragment amino acid residues 173-176 of pHBP
RGTP

SEQ ID NO: 350 - protein, 4 aa, Artificial sequence
  Peptide fragment amino acid residues 177-180 of pHBP
LVCN

SEQ ID NO: 351 - protein, 4 aa, Artificial sequence
  Peptide fragment amino acid residues 178-181 of pHBP
VCNG

SEQ ID NO: 352 - protein, 4 aa, Artificial sequence
  Peptide fragment amino acid residues 179-182 of pHBP
CNGL

SEQ ID NO: 353 - protein, 4 aa, Artificial sequence
  Peptide fragment amino acid residues 180-183 of pHBP
NGLA

SEQ ID NO: 354 - protein, 4 aa, Artificial sequence
  Peptide fragment amino acid residues181-184 of pHBP
GLAQ

SEQ ID NO: 355 - protein, 4 aa, Artificial sequence
  Peptide fragment amino acid residues 182-185 of pHBP
LAQG

SEQ ID NO: 356 - protein, 4 aa, Artificial sequence
  Peptide fragment amino acid residues 183-186 of pHBP
AQGV

SEQ ID NO: 357 - protein, 4 aa, Artificial sequence
  Peptide fragment amino acid residues 184-187 of pHBP
QGVA

SEQ ID NO: 358 - protein, 4 aa, Artificial sequence
  Peptide fragment amino acid residues 187-190 of pHBP
ASFL

SEQ ID NO: 359 - protein, 4 aa, Artificial sequence
  Peptide fragment amino acid residues 188-191 of pHBP
SFLR

SEQ ID NO: 360 - protein, 4 aa, Artificial sequence
  Peptide fragment amino acid residues 189-192 of pHBP
FLRR

SEQ ID NO: 361 - protein, 4 aa, Artificial sequence
  Peptide fragment amino acid residues 19-22 of pHBP
QKQG

SEQ ID NO: 362 - protein, 4 aa, Artificial sequence
  Peptide fragment amino acid residues 191-194 of pHBP
RRRF

SEQ ID NO: 363 - protein, 4 aa, Artificial sequence
  Peptide fragment amino acid residues 192-195 of pHBP
RRFR

SEQ ID NO: 364 - protein, 4 aa, Artificial sequence
  Peptide fragment amino acid residues 193-196 of pHBP
RFRR

SEQ ID NO: 365 - protein, 4 aa, Artificial sequence
  Peptide fragment amino acid residues 194-197 of pHBP
FRRS

SEQ ID NO: 366 - protein, 4 aa, Artificial sequence
  Peptide fragment amino acid residues 195-198 of pHBP
RRSS

SEQ ID NO: 367 - protein, 4 aa, Artificial sequence
  Peptide fragment amino acid residues 196-199 of pHBP
RSSG

SEQ ID NO: 368 - protein, 4 aa, Artificial sequence
  Peptide fragment amino acid residues 197-200 of pHBP
SSGF

SEQ ID NO: 369 - protein, 4 aa, Artificial sequence
  Peptide fragment amino acid residues 198-201 of pHBP
SGFF

SEQ ID NO: 370 - protein, 4 aa, Artificial sequence
  Peptide fragment amino acid residues 199-202 of pHBP
GFFT

SEQ ID NO: 371 - protein, 4 aa, Artificial sequence
  Peptide fragment amino acid residues 20-23 of pHBP
KQGR

SEQ ID NO: 372 - protein, 4 aa, Artificial sequence
  Peptide fragment amino acid residues 206-209 of pHBP
LFRN

SEQ ID NO: 373 - protein, 4 aa, Artificial sequence
  Peptide fragment amino acid residues 207-210 of pHBP
FRNW

SEQ ID NO: 374 - protein, 4 aa, Artificial sequence
  Peptide fragment amino acid residues 208-211 of pHBP
RNWI

SEQ ID NO: 375 - protein, 4 aa, Artificial sequence
  Peptide fragment amino acid residues 209-212 of pHBP
NWID

SEQ ID NO: 376 - protein, 4 aa, Artificial sequence
  Peptide fragment amino acid residues 210-213 of pHBP
WIDS

SEQ ID NO: 377 - protein, 4 aa, Artificial sequence
  Peptide fragment amino acid residues 211-214 of pHBP
IDSV

SEQ ID NO: 378 - protein, 4 aa, Artificial sequence
  Peptide fragment amino acid residues 212-214 of pHBP
DSVL

SEQ ID NO: 379 - protein, 4 aa, Artificial sequence
  Peptide fragment amino acid residues 213-216 of pHBP
SVLN

SEQ ID NO: 380 - protein, 4 aa, Artificial sequence
  Peptide fragment amino acid residues 216-219 of pHBP
NNPP

SEQ ID NO: 381 - protein, 4 aa, Artificial sequence
  Peptide fragment amino acid residues 3-6 of hNLE
GGRR

SEQ ID NO: 382 - protein, 4 aa, Artificial sequence
  Peptide fragment amino acid residues 4-7 of hNLE
GRRA

SEQ ID NO: 383 - protein, 4 aa, Artificial sequence
  Peptide fragment amino acid residues 5-8 of hNLE
RRAR

SEQ ID NO: 384 - protein, 4 aa, Artificial sequence
  Peptide fragment amino acid residues 6-9 of hNLE
RARP

SEQ ID NO: 385 - protein, 4 aa, Artificial sequence
  Peptide fragment amino acid residues 7-10 of hNLE
ARPH

SEQ ID NO: 386 - protein, 4 aa, Artificial sequence
  Peptide fragment amino acid residues 8-11 of hNLE
RPHA

SEQ ID NO: 387 - protein, 4 aa, Artificial sequence
  Peptide fragment amino acid residues 9-12 of hNLE
PHAW

SEQ ID NO: 388 - protein, 4 aa, Artificial sequence
  Peptide fragment amino acid residues 10-13 of hNLE
HAWP

SEQ ID NO: 389 - protein, 4 aa, Artificial sequence
  Peptide fragment amino acid residues 11-14 of hNLE
AWPF

SEQ ID NO: 390 - protein, 4 aa, Artificial sequence
  Peptide fragment amino acid residues 12-15 of hNLE
WPFM

SEQ ID NO: 391 - protein, 4 aa, Artificial sequence
  Peptide fragment amino acid residues 13-16 of hNLE
PFMV

SEQ ID NO: 392 - protein, 4 aa, Artificial sequence
  Peptide fragment amino acid residues 14-17 of hNLE
FMVS

SEQ ID NO: 393 - protein, 4 aa, Artificial sequence
  Peptide fragment amino acid residues 15-18 of hNLE
MVSL

SEQ ID NO: 394 - protein, 4 aa, Artificial sequence
  Peptide fragment amino acid residues 16-19 of hNLE
VSLQ

SEQ ID NO: 395 - protein, 4 aa, Artificial sequence
  Peptide fragment amino acid residues 17-20 of hNLE
SLQL

SEQ ID NO: 396 - protein, 4 aa, Artificial sequence
  Peptide fragment amino acid residues 18-21 of hNLE
LQLR

SEQ ID NO: 397 - protein, 4 aa, Artificial sequence
  Peptide fragment amino acid residues 19-22 of hNLE
QLRG

SEQ ID NO: 398 - protein, 4 aa, Artificial sequence
  Peptide fragment amino acid residues 20-23 of hNLE
LRGG

SEQ ID NO: 399 - protein, 4 aa, Artificial sequence
  Peptide fragment amino acid residues 21-24 of hNLE
RGGH

SEQ ID NO: 400 - protein, 4 aa, Artificial sequence
  Peptide fragment amino acid residues 22-25 of hNLE
GGHF

SEQ ID NO: 401 - protein, 4 aa, Artificial sequence
  Peptide fragment amino acid residues 23-26 of hNLE
GHFC

SEQ ID NO: 402 - protein, 4 aa, Artificial sequence
  Peptide fragment amino acid residues 25-28 of hNLE
FCGA

SEQ ID NO: 403 - protein, 4 aa, Artificial sequence
  Peptide fragment amino acid residues 26-29 of hNLE
CGAT

SEQ ID NO: 404 - protein, 4 aa, Artificial sequence
  Peptide fragment amino acid residues 27-30 of hNLE
GATL

SEQ ID NO: 405 - protein, 4 aa, Artificial sequence
  Peptide fragment amino acid residues 28-31 of hNLE
ATLI

SEQ ID NO: 406 - protein, 4 aa, Artificial sequence
  Peptide fragment amino acid residues 29-32 of hNLE
TLIA

SEQ ID NO: 407 - protein, 4 aa, Artificial sequence
  Peptide fragment amino acid residues 30-33 of hNLE
LIAP

SEQ ID NO: 408 - protein, 4 aa, Artificial sequence
  Peptide fragment amino acid residues 31-34 of hNLE
IAPN

SEQ ID NO: 409 - protein, 4 aa, Artificial sequence
  Peptide fragment amino acid residues 32-35 of hNLE
APNF

SEQ ID NO: 410 - protein, 4 aa, Artificial sequence
  Peptide fragment amino acid residues 32-36 of hNLE
PNFV

SEQ ID NO: 411 - protein, 4 aa, Artificial sequence
  Peptide fragment amino acid residues 34-37 of hNLE
NFVM

SEQ ID NO: 412 - protein, 4 aa, Artificial sequence
  Peptide fragment amino acid residues 35-38 of hNLE
FVMS

SEQ ID NO: 413 - protein, 4 aa, Artificial sequence
  Peptide fragment amino acid residues 36-39 of hNLE
VMSA

SEQ ID NO: 414 - protein, 4 aa, Artificial sequence
  Peptide fragment amino acid residues 37-40 of hNLE
MSAA

SEQ ID NO: 415 - protein, 4 aa, Artificial sequence
  Peptide fragment amino acid residues 38-41of hNLE
SAAH

SEQ ID NO: 416 - protein, 4 aa, Artificial sequence
  Peptide fragment amino acid residues 39-42 of hNLE
AAHC

SEQ ID NO: 417 - protein, 4 aa, Artificial sequence
  Peptide fragment amino acid residues 40-43 of hNLE
AHCV

SEQ ID NO: 418 - protein, 4 aa, Artificial sequence
  Peptide fragment amino acid residues 41-44 of hNLE
HCVA

SEQ ID NO: 419 - protein, 4 aa, Artificial sequence
  Peptide fragment amino acid residues 42-45 of hNLE
CVAN

SEQ ID NO: 420 - protein, 4 aa, Artificial sequence
  Peptide fragment amino acid residues 43-36 of hNLE
VANV

SEQ ID NO: 421 - protein, 4 aa, Artificial sequence
  Peptide fragment amino acid residues 44-47 of hNLE
ANVN

SEQ ID NO: 422 - protein, 4 aa, Artificial sequence
  Peptide fragment amino acid residues 45-48 of hNLE
NVNV

SEQ ID NO: 423 - protein, 4 aa, Artificial sequence
  Peptide fragment amino acid residues 46-49 of hNLE
VNVR

SEQ ID NO: 424 - protein, 4 aa, Artificial sequence
  Peptide fragment amino acid residues 48-51 of hNLE
VRAV

SEQ ID NO: 425 - protein, 4 aa, Artificial sequence
  Peptide fragment amino acid residues 49-52 of hNLE
RAVR

SEQ ID NO: 426 - protein, 4 aa, Artificial sequence
  Peptide fragment amino acid residues 50-53 of hNLE
AVRV

SEQ ID NO: 427 - protein, 4 aa, Artificial sequence
  Peptide fragment amino acid residues 51-54 of hNLE
VRVV

SEQ ID NO: 428 - protein, 4 aa, Artificial sequence
  Peptide fragment amino acid residues 52-55 of hNLE
RVVL

SEQ ID NO: 429 - protein, 4 aa, Artificial sequence
  Peptide fragment amino acid residues 55-58 of hNLE
LGAH

SEQ ID NO: 430 - protein, 4 aa, Artificial sequence
  Peptide fragment amino acid residues 56-59 of hNLE
GAHN

SEQ ID NO: 431 - protein, 4 aa, Artificial sequence
  Peptide fragment amino acid residues 57-60 of hNLE
AHNL

SEQ ID NO: 432 - protein, 4 aa, Artificial sequence
  Peptide fragment amino acid residues 58-61 of hNLE
HNLS

SEQ ID NO: 433 - protein, 4 aa, Artificial sequence
  Peptide fragment amino acid residues 59-62 of hNLE
NLSR

SEQ ID NO: 434 - protein, 4 aa, Artificial sequence
  Peptide fragment amino acid residues 60-63 of hNLE
LSRR

SEQ ID NO: 435 - protein, 4 aa, Artificial sequence
  Peptide fragment amino acid residues 61-64 of hNLE
SRRE

SEQ ID NO: 436 - protein, 4 aa, Artificial sequence
  Peptide fragment amino acid residues 62-65 of hNLE
RREP

SEQ ID NO: 437 - protein, 4 aa, Artificial sequence
  Peptide fragment amino acid residues 63-66 of hNLE
REPT

SEQ ID NO: 438 - protein, 4 aa, Artificial sequence
  Peptide fragment amino acid residues 64-67 of hNLE
EPTR

SEQ ID NO: 439 - protein, 4 aa, Artificial sequence
  Peptide fragment amino acid residues 65-68 of hNLE
PTRQ

SEQ ID NO: 440 - protein, 4 aa, Artificial sequence
  Peptide fragment amino acid residues 66-69 of hNLE
TRQV

SEQ ID NO: 441 - protein, 4 aa, Artificial sequence
  Peptide fragment amino acid residues 67-70 of hNLE
RQVF

SEQ ID NO: 442 - protein, 4 aa, Artificial sequence
  Peptide fragment amino acid residues 68-71 of hNLE
QVFA

SEQ ID NO: 443 - protein, 4 aa, Artificial sequence
  Peptide fragment amino acid residues 69-72 of hNLE
VFAV

SEQ ID NO: 444 - protein, 4 aa, Artificial sequence
  Peptide fragment amino acid residues 70-73 of hNLE
FAVQ

SEQ ID NO: 445 - protein, 4 aa, Artificial sequence
  Peptide fragment amino acid residues 71-74 of hNLE
AVQR

SEQ ID NO: 446 - protein, 4 aa, Artificial sequence
  Peptide fragment amino acid residues 72-75 of hNLE
VQRI

SEQ ID NO: 447 - protein, 4 aa, Artificial sequence
  Peptide fragment amino acid residues 73-76 of hNLE
QRIF

SEQ ID NO: 448 - protein, 4 aa, Artificial sequence
  Peptide fragment amino acid residues 74-77 of hNLE
RIFE

SEQ ID NO: 449 - protein, 4 aa, Artificial sequence
  Peptide fragment amino acid residues 75-78 of hNLE
IFED

SEQ ID NO: 450 - protein, 4 aa, Artificial sequence
  Peptide fragment amino acid residues 76-79 of hNLE
FEDG

SEQ ID NO: 451 - protein, 4 aa, Artificial sequence
  Peptide fragment amino acid residues 77-80 of hNLE
EDGY

SEQ ID NO: 452 - protein, 4 aa, Artificial sequence
  Peptide fragment amino acid residues 78-81 of hNLE
DGYD

SEQ ID NO: 453 - protein, 4 aa, Artificial sequence
  Peptide fragment amino acid residues 80-83 of hNLE
YDPV

SEQ ID NO: 454 - protein, 4 aa, Artificial sequence
  Peptide fragment amino acid residues 81-84 of hNLE
DPVN

SEQ ID NO: 455 - protein, 4 aa, Artificial sequence
  Peptide fragment amino acid residues 82-84 of hNLE
PVNL

SEQ ID NO: 456 - protein, 4 aa, Artificial sequence
  Peptide fragment amino acid residues 83-86 of hNLE
VNLL

SEQ ID NO: 457 - protein, 4 aa, Artificial sequence
  Peptide fragment amino acid residues 84-87 of hNLE
NLLN

SEQ ID NO: 458 - protein, 4 aa, Artificial sequence
  Peptide fragment amino acid residues 85-88 of hNLE
LLND

SEQ ID NO: 459 - protein, 4 aa, Artificial sequence
  Peptide fragment amino acid residues 86-89 of hNLE
LNDI

SEQ ID NO: 460 - protein, 4 aa, Artificial sequence
  Peptide fragment amino acid residues 87-90 of hNLE
NDIV

SEQ ID NO: 461 - protein, 4 aa, Artificial sequence
  Peptide fragment amino acid residues 88-91 of hNLE
DIVI

SEQ ID NO: 462 - protein, 4 aa, Artificial sequence
  Peptide fragment amino acid residues 89-92 of hNLE
IVIL

SEQ ID NO: 463 - protein, 4 aa, Artificial sequence
  Peptide fragment amino acid residues 90-93 of hNLE
VILQ

SEQ ID NO: 464 - protein, 4 aa, Artificial sequence
  Peptide fragment amino acid residues 91-94 of hNLE
ILQL

SEQ ID NO: 465 - protein, 4 aa, Artificial sequence
  Peptide fragment amino acid residues 92-95 of hNLE
LQLN

SEQ ID NO: 466 - protein, 4 aa, Artificial sequence
  Peptide fragment amino acid residues 93-96 of hNLE
QLNG

SEQ ID NO: 467 - protein, 4 aa, Artificial sequence
  Peptide fragment amino acid residues 94-97 of hNLE
LNGS

SEQ ID NO: 468 - protein, 4 aa, Artificial sequence
  Peptide fragment amino acid residues 95-98 of hNLE
NGSA

SEQ ID NO: 469 - protein, 4 aa, Artificial sequence
  Peptide fragment amino acid residues 96-99 of hNLE
GSAT

SEQ ID NO: 470 - protein, 4 aa, Artificial sequence
  Peptide fragment amino acid residues 97-100 of hNLE
SATI

SEQ ID NO: 471 - protein, 4 aa, Artificial sequence
  Peptide fragment amino acid residues 98-101 of hNLE
ATIN

SEQ ID NO: 472 - protein, 4 aa, Artificial sequence
  Peptide fragment amino acid residues 99-102 of hNLE
TINP

SEQ ID NO: 473 - protein, 4 aa, Artificial sequence
  Peptide fragment amino acid residues 100-103 of hNLE
INPS

SEQ ID NO: 474 - protein, 4 aa, Artificial sequence
  Peptide fragment amino acid residues 101-104 of hNLE
NPSV

SEQ ID NO: 475 - protein, 4 aa, Artificial sequence
  Peptide fragment amino acid residues 102-105 of hNLE
PSVA

SEQ ID NO: 476 - protein, 4 aa, Artificial sequence
  Peptide fragment amino acid residues 103-106 of hNLE
SVAL

SEQ ID NO: 477 - protein, 4 aa, Artificial sequence
  Peptide fragment amino acid residues 104-107 of hNLE
VALV

SEQ ID NO: 478 - protein, 4 aa, Artificial sequence
  Peptide fragment amino acid residues 105-108 of hNLE
ALVP

SEQ ID NO: 479 - protein, 4 aa, Artificial sequence
  Peptide fragment amino acid residues 106-109 of hNLE
LVPL

SEQ ID NO: 480 - protein, 4 aa, Artificial sequence
  Peptide fragment amino acid residues 107-110 of hNLE
VPLP

SEQ ID NO: 481 - protein, 4 aa, Artificial sequence
  Peptide fragment amino acid residues 108-111 of hNLE
PLPA

SEQ ID NO: 482 - protein, 4 aa, Artificial sequence
  Peptide fragment amino acid residues 109-112 of hNLE
LPAQ

SEQ ID NO: 483 - protein, 4 aa, Artificial sequence
  Peptide fragment amino acid residues 110-113 of hNLE
PAQG

SEQ ID NO: 484 - protein, 4 aa, Artificial sequence
  Peptide fragment amino acid residues 111-114 of hNLE
AQGR

SEQ ID NO: 485 - protein, 4 aa, Artificial sequence
  Peptide fragment amino acid residues 112-115 of hNLE
QGRR

SEQ ID NO: 486 - protein, 4 aa, Artificial sequence
  Peptide fragment amino acid residues 113-116 of hNLE
GRRL

SEQ ID NO: 487 - protein, 4 aa, Artificial sequence
  Peptide fragment amino acid residues 114-117 of hNLE
RRLG

SEQ ID NO: 488 - protein, 4 aa, Artificial sequence
  Peptide fragment amino acid residues 115-118 of hNLE
RLGN

SEQ ID NO: 489 - protein, 4 aa, Artificial sequence
  Peptide fragment amino acid residues 116-119 of hNLE
LGNG

SEQ ID NO: 490 - protein, 4 aa, Artificial sequence
  Peptide fragment amino acid residues 117-120 of hNLE
GNGV

SEQ ID NO: 491 - protein, 4 aa, Artificial sequence
  Peptide fragment amino acid residues 118-121 of hNLE
NGVQ

SEQ ID NO: 492 - protein, 4 aa, Artificial sequence
  Peptide fragment amino acid residues 119-121 of hNLE
GVQC

SEQ ID NO: 493 - protein, 4 aa, Artificial sequence
  Peptide fragment amino acid residues 120-123 of hNLE
VQCL

SEQ ID NO: 494 - protein, 4 aa, Artificial sequence
  Peptide fragment amino acid residues 121-124 of hNLE
QCLA

SEQ ID NO: 495 - protein, 4 aa, Artificial sequence
  Peptide fragment amino acid residues 122-125 of hNLE
CLAM

SEQ ID NO: 496 - protein, 4 aa, Artificial sequence
  Peptide fragment amino acid residues 123-126 of hNLE
LAMG

SEQ ID NO: 497 - protein, 4 aa, Artificial sequence
  Peptide fragment amino acid residues 124-127 of hNLE
AMGW

SEQ ID NO: 498 - protein, 4 aa, Artificial sequence
  Peptide fragment amino acid residues 125-128 of hNLE
MGWG

SEQ ID NO: 499 - protein, 4 aa, Artificial sequence
  Peptide fragment amino acid residues 126-129 of hNLE
GWGL

SEQ ID NO: 500 - protein, 4 aa, Artificial sequence
  Peptide fragment amino acid residues 127-130 of hNLE
WGLL

SEQ ID NO: 501 - protein, 4 aa, Artificial sequence
  Peptide fragment amino acid residues 47-50 of hNLE
NVRA

SEQ ID NO: 502 - protein, 4 aa, Artificial sequence
  Peptide fragment amino acid residues 128-131of hNLE
GLLG

SEQ ID NO: 503 - protein, 4 aa, Artificial sequence
  Peptide fragment amino acid residues 129-132 of hNLE
LLGR

SEQ ID NO: 504 - protein, 4 aa, Artificial sequence
  Peptide fragment amino acid residues 130-133 of hNLE
LGRN

SEQ ID NO: 505 - protein, 4 aa, Artificial sequence
  Peptide fragment amino acid residues 131-134 of hNLE
GRNR

SEQ ID NO: 506 - protein, 4 aa, Artificial sequence
  Peptide fragment amino acid residues 132-135 of hNLE
RNRG

SEQ ID NO: 507 - protein, 4 aa, Artificial sequence
  Peptide fragment amino acid residues 133-136 of hNLE
NRGI

SEQ ID NO: 508 - protein, 4 aa, Artificial sequence
  Peptide fragment amino acid residues 134-137 of hNLE
RGIA

SEQ ID NO: 509 - protein, 4 aa, Artificial sequence
  Peptide fragment amino acid residues 135-138 of hNLE
GIAS

SEQ ID NO: 510 - protein, 4 aa, Artificial sequence
  Peptide fragment amino acid residues 136-139 of hNLE
IASV

SEQ ID NO: 511 - protein, 4 aa, Artificial sequence
  Peptide fragment amino acid residues 137-140 of hNLE
ASVL

SEQ ID NO: 512 - protein, 4 aa, Artificial sequence
  Peptide fragment amino acid residues 138-141 of hNLE
SVLQ

SEQ ID NO: 513 - protein, 4 aa, Artificial sequence
  Peptide fragment amino acid residues 139-142 of hNLE
VLQE

SEQ ID NO: 514 - protein, 4 aa, Artificial sequence
  Peptide fragment amino acid residues 140-143 of hNLE
LQEL

SEQ ID NO: 515 - protein, 4 aa, Artificial sequence
  Peptide fragment amino acid residues 141-144 of hNLE
QELN

SEQ ID NO: 516 - protein, 4 aa, Artificial sequence
  Peptide fragment amino acid residues 142-145 of hNLE
ELNV

SEQ ID NO: 517 - protein, 4 aa, Artificial sequence
  Peptide fragment amino acid residues 143-146 of hNLE
LNVT

SEQ ID NO: 518 - protein, 4 aa, Artificial sequence
  Peptide fragment amino acid residues 145-148 of hNLE
VTVV

SEQ ID NO: 519 - protein, 4 aa, Artificial sequence
  Peptide fragment amino acid residues 146-149 of hNLE
TVVT

SEQ ID NO: 520 - protein, 4 aa, Artificial sequence
  Peptide fragment amino acid residues 147-150 of hNLE
VVTS

SEQ ID NO: 521 - protein, 4 aa, Artificial sequence
  Peptide fragment amino acid residues 148-151 of hNLE
VTSL

SEQ ID NO: 522 - protein, 4 aa, Artificial sequence
  Peptide fragment amino acid residues 149-152 of hNLE
TSLC

SEQ ID NO: 523 - protein, 4 aa, Artificial sequence
  Peptide fragment amino acid residues 150-153 of hNLE
SLCR

SEQ ID NO: 524 - protein, 4 aa, Artificial sequence
  Peptide fragment amino acid residues 151-154 of hNLE
LCRR

SEQ ID NO: 525 - protein, 4 aa, Artificial sequence
  Peptide fragment amino acid residues 152-155 of hNLE
CRRS

SEQ ID NO: 526 - protein, 4 aa, Artificial sequence
  Peptide fragment amino acid residues 153-156 of hNLE
RRSN

SEQ ID NO: 527 - protein, 4 aa, Artificial sequence
  Peptide fragment amino acid residues 154-157 of hNLE
RSNV

SEQ ID NO: 528 - protein, 4 aa, Artificial sequence
  Peptide fragment amino acid residues 155-158 of hNLE
SNVC

SEQ ID NO: 529 - protein, 4 aa, Artificial sequence
  Peptide fragment amino acid residues 157-160 of hNLE
VCTL

SEQ ID NO: 530 - protein, 4 aa, Artificial sequence
  Peptide fragment amino acid residues 158-161 of hNLE
CTLV

SEQ ID NO: 531 - protein, 4 aa, Artificial sequence
  Peptide fragment amino acid residues 159-162 of hNLE
TLVR

SEQ ID NO: 532 - protein, 4 aa, Artificial sequence
  Peptide fragment amino acid residues 160-163 of hNLE
LVRG

SEQ ID NO: 533 - protein, 4 aa, Artificial sequence
  Peptide fragment amino acid residues 161-164 of hNLE
VRGR

SEQ ID NO: 534 - protein, 4 aa, Artificial sequence
  Peptide fragment amino acid residues 162-165 of hNLE
RGRR

SEQ ID NO: 535 - protein, 4 aa, Artificial sequence
  Peptide fragment amino acid residues 163-166 of hNLE
GRRG

SEQ ID NO: 536 - protein, 4 aa, Artificial sequence
  Peptide fragment amino acid residues 164-167 of hNLE
RRGR

SEQ ID NO: 537 - protein, 4 aa, Artificial sequence
  Peptide fragment amino acid residues 165-166 of hNLE
RGRI

SEQ ID NO: 538 - protein, 4 aa, Artificial sequence
  Peptide fragment amino acid residues 166-169 of hNLE
GRIS

SEQ ID NO: 539 - protein, 4 aa, Artificial sequence
  Peptide fragment amino acid residues 167-170 of hNLE
RISQ

SEQ ID NO: 540 - protein, 4 aa, Artificial sequence
  Peptide fragment amino acid residues 168-171of hNLE
ISQG

SEQ ID NO: 541 - protein, 4 aa, Artificial sequence
  Peptide fragment amino acid residues 169-172 of hNLE
SQGD

SEQ ID NO: 542 - protein, 4 aa, Artificial sequence
  Peptide fragment amino acid residues 170-173 of hNLE
QGDS

SEQ ID NO: 543 - protein, 4 aa, Artificial sequence
  Peptide fragment amino acid residues 171-174 of hNLE
GDSG

SEQ ID NO: 544 - protein, 4 aa, Artificial sequence
  Peptide fragment amino acid residues 172-174 of hNLE
DSGT

SEQ ID NO: 545 - protein, 4 aa, Artificial sequence
  Peptide fragment amino acid residues 173-176 of hNLE
SGTP

SEQ ID NO: 546 - protein, 4 aa, Artificial sequence
  Peptide fragment amino acid residues 177-180 of hNLE
LVCN

SEQ ID NO: 547 - protein, 4 aa, Artificial sequence
  Peptide fragment amino acid residues 178-181 of hNLE
VCNG

SEQ ID NO: 548 - protein, 4 aa, Artificial sequence
  Peptide fragment amino acid residues 179-182 of hNLE
CNGL

SEQ ID NO: 549 - protein, 4 aa, Artificial sequence
  Peptide fragment amino acid residues 180-183 of hNLE
NGLI

SEQ ID NO: 550 - protein, 4 aa, Artificial sequence
  Peptide fragment amino acid residues 181-184 of hNLE
GLIH

SEQ ID NO: 551 - protein, 4 aa, Artificial sequence
  Peptide fragment amino acid residues 182-185 of hNLE
LIHG

SEQ ID NO: 552 - protein, 4 aa, Artificial sequence
  Peptide fragment amino acid residues 183-186 of hNLE
IHGI

SEQ ID NO: 553 - protein, 4 aa, Artificial sequence
  Peptide fragment amino acid residues 184-187 of hNLE
HGIA

SEQ ID NO: 554 - protein, 4 aa, Artificial sequence
  Peptide fragment amino acid residues 185-188 of hNLE
GIAS

SEQ ID NO: 555 - protein, 4 aa, Artificial sequence
  Peptide fragment amino acid residues 186-189 of hNLE
IASF

SEQ ID NO: 556 - protein, 4 aa, Artificial sequence
  Peptide fragment amino acid residues 187-190 of hNLE
ASFV

SEQ ID NO: 557 - protein, 4 aa, Artificial sequence
  Peptide fragment amino acid residues 188-191 of hNLE
SFVR

SEQ ID NO: 558 - protein, 4 aa, Artificial sequence
  Peptide fragment amino acid residues 189-192 of hNLE
FVRG

SEQ ID NO: 559 - protein, 4 aa, Artificial sequence
  Peptide fragment amino acid residues 190-103 of hNLE
VRGG

SEQ ID NO: 560 - protein, 4 aa, Artificial sequence
  Peptide fragment amino acid residues 191-194 of hNLE
RGGC

SEQ ID NO: 561 - protein, 4 aa, Artificial sequence
  Peptide fragment amino acid residues 192-195 of hNLE
GGCA

SEQ ID NO: 562 - protein, 4 aa, Artificial sequence
  Peptide fragment amino acid residues 193-196 of hNLE
GCAS

SEQ ID NO: 563 - protein, 4 aa, Artificial sequence
  Peptide fragment amino acid residues 194-197 of hNLE
CASG

SEQ ID NO: 564 - protein, 4 aa, Artificial sequence
  Peptide fragment amino acid residues 195-198 of hNLE
ASGL

SEQ ID NO: 565 - protein, 4 aa, Artificial sequence
  Peptide fragment amino acid residues 196-199 of hNLE
SGLY

SEQ ID NO: 566 - protein, 4 aa, Artificial sequence
  Peptide fragment amino acid residues 197-200 of hNLE
GLYP

SEQ ID NO: 567 - protein, 4 aa, Artificial sequence
  Peptide fragment amino acid residues 198-201 of hNLE
LYPD

SEQ ID NO: 568 - protein, 4 aa, Artificial sequence
  Peptide fragment amino acid residues 199-202 of hNLE
YPDA

SEQ ID NO: 569 - protein, 4 aa, Artificial sequence
  Peptide fragment amino acid residues 200-203 of hNLE
PDAF

SEQ ID NO: 570 - protein, 4 aa, Artificial sequence
  Peptide fragment amino acid residues 201-204 of hNLE
DAFA

SEQ ID NO: 571 - protein, 4 aa, Artificial sequence
  Peptide fragment amino acid residues 202-205 of hNLE
AFAP

SEQ ID NO: 572 - protein, 4 aa, Artificial sequence
  Peptide fragment amino acid residues 203-206 of hNLE
FAPV

SEQ ID NO: 573 - protein, 4 aa, Artificial sequence
  Peptide fragment amino acid residues 204-207 of hNLE
APVA

SEQ ID NO: 574 - protein, 4 aa, Artificial sequence
  Peptide fragment amino acid residues 205-208 of hNLE
PVAQ

SEQ ID NO: 575 - protein, 4 aa, Artificial sequence
  Peptide fragment amino acid residues 206-209 of hNLE
VAQF

SEQ ID NO: 576 - protein, 4 aa, Artificial sequence
  Peptide fragment amino acid residues 207-210 of hNLE
AQFV

SEQ ID NO: 577 - protein, 4 aa, Artificial sequence
  Peptide fragment amino acid residues 208-211 of hNLE
QFVN

SEQ ID NO: 578 - protein, 4 aa, Artificial sequence
  Peptide fragment amino acid residues 209-212 of hNLE
FVNW

SEQ ID NO: 579 - protein, 4 aa, Artificial sequence
  Peptide fragment amino acid residues 210-212 of hNLE
VNWI

SEQ ID NO: 580 - protein, 4 aa, Artificial sequence
  Peptide fragment amino acid residues 211-214 of hNLE
NWID

SEQ ID NO: 581 - protein, 4 aa, Artificial sequence
  Peptide fragment amino acid residues 212-215 of hNLE
WIDS

SEQ ID NO: 582 - protein, 4 aa, Artificial sequence
  Peptide fragment amino acid residues 213-216 of hNLE
IDSI

SEQ ID NO: 583 - protein, 4 aa, Artificial sequence
  Peptide fragment amino acid residues 214-217 of hNLE
DSII

SEQ ID NO: 584 - protein, 4 aa, Artificial sequence
  Peptide fragment amino acid residues 214-218 of hNLE
SIIQ

SEQ ID NO: 585 - protein, 4 aa, Artificial sequence
  Peptide fragment amino acid residues 43-46 of pHBP
PRFV

SEQ ID NO: 586 - protein, 4 aa, Artificial sequence
  Peptide fragment amino acid residues 190-194 of pHBP
LRRR

SEQ ID NO: 587 - protein, 4 aa, Artificial sequence
  Peptide fragment amino acid residues 217-220 of pHBP
NPPA

SEQ ID NO: 588 - protein, 221 aa, Sus sp.
IVGGRRAQPQEFPFLASIQKQGRPFCAGALVHPRFVLTAASCFRGKNSGSASVVLGAYDLRQQEQSRQTFSIRSISQNGYDPRQNLNDVLLLQLDREARLTPSVALVPLPPQNATVEAGTNCQVEAGWGTQRLRRLFSRFPRVLNVTVTSNPCLPRDMCIGVFSRRGRISQGDRGTPLVCNGLAQGVASFLRRRFRRSSGFFTRVALFRNWIDSVLNNPPA

SEQ ID NO: 589 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH